(19) United States
(12) Patent Application Publication
(10) Pub. No.: US 2017/0189357 A1
(43) Pub. Date: Jul. 6, 2017

(54) METHODS OF LIMITING MORBIDITY IN HEMOGLOBINOPATHIES

(71) Applicant: DUKE UNIVERSITY, Durham, NC (US)

(72) Inventor: Rahima Zennadi, Durham, NC (US)

(73) Assignee: Duke University, Durham, NC (US)

(21) Appl. No.: 15/313,689

(22) PCT Filed: May 26, 2015
(86) PCT No.: PCT/US2015/032366

§ 371 Date: Nov. 23, 2016

Related U.S. Application Data
(60) Provisional application No. 62/002,288, filed on May 23, 2014.

(51) Int. Cl. A61K 31/18; G01N 33/573

(52) U.S. Cl. CPC A61K 31/18; G01N 33/573; G01N 2800/52

(57) ABSTRACT

Methods of alleviating the symptoms of hemoglobinopathies, including, but not limited to, sickle cell disease, β-thalassemia, and hemoglobin H disease are provided. In some embodiments, the methods comprise administering an agent to the subject if the subject has increased expression or activation of at least one of ERK, Ras, BRAF, Raf1 MEK, β-arrest1/2, Syk, P60-c-Src, or GRK2. Methods of determining the likelihood of a complication or vascular endothelial injury and mortality resulting from a hemoglobinopathy in a subject are also provided.

### CROSS-REFERENCE TO RELATED APPLICATIONS

This patent application claims the benefit of priority of U.S. Provisional Patent Application No. 62/002,288, filed May 23, 2014, which is incorporated herein by reference in its entirety.

### STATEMENT REGARDING FEDERALLY SPONSORED RESEARCH

This invention was made with government support under K01-DK065040 awarded by the National institutes of Health: National institute of Diabetes and Digestive and Kidney Diseases. The government has certain rights in the invention.

### BACKGROUND

Vaso-occlusive phenomena and hemolytic anemia are the clinical hallmarks of sickle cell disease (SCD). Sickle red blood cell (RBC)-based adhesion and vaso-occlusive events likely initiate and/or exacerbate the profound vasculopathy present in SCD. Vase-occlusion results in recurrent painful episodes and a variety of serious organ system complications that can lead to life-long disabilities and even death. Episodic vase-occlusion causes the painful crises that are familiar to almost all SCD patients. Vase-occlusion is unpredictable and is thought to be responsible for most organ damage and reduced life expectancy in adults with SCD. Studies have tried to relate markers of inflammation and endothelial injury to specific outcomes. Several of these markers are elevated during vase-occlusive crisis. However, none of the markers reliably predicts occurrence of vaso-occlusion. The direct cause of and the explanations for varying vase-occlusion leading to vascular damage among patients are still unclear. Sickle RBCs are central to vaso-occlusion and the associated vascular damage, and the relation of sickle RBC signaling sequelae to cell adhesion-associated vascular injury is unknown.

Sickle RBCs possess unusually active signaling pathways that contribute to a panopoly of abnormalities, including RBC adhesion to the endothelium, vaso-occlusion and vascular injury.^{2, 16, 17} Cell adhesion is a multistep cellular process that is regulated by complex extracellular and intracellular signals, which may differ from one cell type to another. We have previously shown that abnormal sickle RBC interactions with the endothelium and with leukocytes can be increased via stimulation of β₂ adrenergic receptors (ARs) by the stress hormone epinephrine.^{17, 19} Such stimulation activates the intracellular cyclic adenosine monophosphate (cAMP)/protein kinase A (PKA) pathway.¹⁷ βARs are prototypic G protein-coupled receptors (GPCRs), whose signaling properties are largely mediated by activation of stimulatory GTP-binding proteins (Gs proteins), which in turn activate adenylate cyclase (AC), leading to generation of cAMP, and the subsequent activation of PKA. The cAMP/PKA pathway can modulate the mitogen-activated protein kinase MAPK)/extracellular signal-regulated kinase (ERKs) cascade.²⁰ PKA has been reported to stimulate B-Raf, while inhibiting c-Raf. Therefore, the activity of downstream signaling proteins, such as MEKs and ERKs, could be either enhanced or inhibited depending on the balance of c-Raf and B-Raf activation.^{21, 22} The cellular functions mediated by βARs can also be independent of adenylyl cyclase activation and involve other mediators instead.^{23, 24}

There is a growing body of evidence showing that activated monocytes²⁵ and neutrophils²⁶ also adhere to the vascular endothelium and contribute to the vasoocclusive processes in SCD. In sickle mice, murine sickle cells bind to adherent leukocytes in inflamed cremasteric vessels producing vasoocclusion.^{¢}It has been also shown that ERK pathway inhibition in neutrophils down-regulates adhesion molecule expression induced by endothelin-1, and cell adhesive response in vitro.²⁸

### SUMMARY

Of the hemoglobinopathies, sickle cell disease and β-thalassemia have the highest impact on morbidity and mortality. Both are prototypical Mendelian single gene disorders affecting the β-globin (HBB) gene. Despite a simple genetic basis, these disorders display extreme clinical, heterogeneity. For instance, in SCD, while some patients have only sporadic pain crises with few if any long-term complications, others experience serious crises with multiple long term complications, high levels of morbidity and accelerated mortality. These distinct clinical outcomes are not well understood. The exact mechanisms that predispose adults to develop vaso-occlusion are unknown. In addition, several markers are elevated during vaso-occlusive crisis. However, none of the markers reliably predicts occurrence of vaso-occlusion. Because sickle RBCs are central to vaso-occlusion, the relation of sickle RBC signaling sequelae to cell adhesion-associated endothelial dysfunction and vascular injury is novel and needs to be addressed. Identification of valuable biomarkers of sickle RBC signaling pathways may be useful to predict complications and inform new therapies to avert organ damage.

In some embodiments, methods of alleviating at least one symptom of a hemoglobinopathy in a subject are provided. The methods include obtaining a sample including red blood cells from a subject and optionally treating the red blood cells with at least one of cholera toxin, pertussis toxin, TNF-α, epinephrine or exposing the cells to hypoxia. The level of expression, activation or membrane translocation of at least one marker selected from ERK 1/2 (ERK), Ras, BRAF, Raf1, MEK 1/2 (MEK), β-arrestin1/2, Syk, p60-c-Src, or GRK2 in the sample is determined and evaluated or compared to a reference level or control. The comparison or evaluation can then be used to develop a treatment plan for the subject. In subjects displaying increased expression, activation or membrane translocation of the at least one marker, an agent capable of inhibiting at least one symptom of the hemoglobinopathy is administered to the subject. In one embodiment, the agent is administered if the expression, activation or membrane translocation of at least one of ERK, Ras, BRAF/Raf1, MEK, β-arrestin1/2, Syk, p60-c-Src or GRK2 is above that of control cells or above a reference level indicative of increased likelihood or severity of a symptom of a hemoglobinopathy. In some embodiments, a hemoglobinopathy is selected from sickle cell disease, β-thalassemia, and hemoglobin H disease. In some embodiments, at least one symptom is selected from vaso-occlusion, acute or chronic painful episodes, chronic hemolysis (aplastic crises), vascular dysfunction and injury, avascular necrosis, infection, end-organ damage, and erythroid hyperplasia.

In a further aspect, methods of determining the severity of sickle cell disease or another hemolobinopathy are provided. The methods include obtaining a blood sample including red blood cells from a subject and optionally treating the red blood cells with at least one of cholera toxin, pertussis toxin, TNF-α, epinephrine or exposing the cells to hypoxia. The cells are then assessed for expression, activation or membrane translocation of at least one of ERK, Ras, BRAF, Raf1, MEK, β-arrestin1/2, Syk, p60-c-Src or GRK2. Suitably, in this step, the cells are assessed for at least one of ERK phosphorylation and expression, MEK phosphorylation and expression, GRK2 expression and membrane translocation, and phosphorylation, or β-arrestin1/2 expression and membrane translocation, and phosphorylation. The expression and/or activation levels of these markers, such as the level of ERK phosphorylation and expression, MEK phosphorylation and expression, GRK2 membrane translocation, phosphorylation and expression, or β-arrestin1/2 membrane translocation, phosphorylation and expression, is related to the severity of sickle cell disease and/or the likelihood of the red blood cells to adhere to other cells and/or to increased endothelial dysfunction and vascular injury or mortality for the subject.

In a still further aspect, methods of treating at least one symptom of a hemoglobinopathy in a subject are provided. The methods include having an expression, activation or membrane translocation level of at least one marker selected from ERK, Ras, BRAE/Raf1, MEK, β-arrestin1/2, Syk, p60-c-Src, or GRK2 determined in the sample. Based on these levels a treatment regimen for the subject is selected. The final step includes administering a therapeutically effective amount of an agent capable of inhibiting at least one symptom of the hemoglobinopathy to the subject if the expression an for activation, and/or membrane-translocation of at least one of ERK, Ras, BRAF/Raf1 MEK, β-arrestin1/2, Syk, p60-c-Src, or GRK2 is above that of control cells.

### DESCRIPTION OF THE FIGURES

FIG. 1 is a graph showing that the ERK activity level affects sickle RBC adhesion to endothelial cells in vitro. Sickle RBC adhesion to non-activated and TNFα-activated HUVECs was tested in intermittent flow conditions, and results are presented as % adherent sickle RBCs at a shear stress of 2 dynes/cm². Sickle RBCs (n=8) were sham-treated, or treated with the MEK inhibitor U0126 at 10 μM. U0126 significantly inhibited sickle RBC adherence to activated endothelial cells (ECs), and this inhibitory effect varied among patients. p<0.05 compared to sham-treated sickle RBC adherent to non-activated ECs. Error bars show SEM of 3 different experiments.

FIG. 2 is a Western blot and graph showing that ERK is activated in sickle cells via Ras, BRAF/Raf1 and MEK and its activity is up-regulated by hypoxia to mediate adhesion of sickle cells to the endothelium. FIG. 2A shows that sickle cell ERK is activated by hypoxia and acts downstream of Ras, Raf and MEK. Sickle RBCs were sham-treated (lanes 1 and 2), or treated with the MEK inhibitor (MEK I; lanes 3 and 4), Ras inhibitor (Ras I; lane 5) or Raf1 inhibitor (Raf1 BRAF I; lane 6); followed by no exposure (lanes 1 and 4 or exposure for 2 hours to hypoxia (8% oxygen) (lanes 2, 3, 5 and 6). Sickle RBC membrane ghosts were blotted for the total ERK and phosphorylated ERK. Blots from 2 different SCD patients (1) and (2) are shown. Quantitative analysis of the data (normalized according to total ERK) is presented as fold change in ERK phosphorylation. * and ***: p<0.05 compared to non-treated sickle RBCs (lane 1), and **: p<0.001 compared to cells exposed to hypoxia (lane 2). Error bars show SEM of 4 different experiments. FIG. 2B is a graph showing that sickle cell ERK activation is enhanced by hypoxia to mediate adhesion to the endothelium. Sickle cells were exposed to room air (sham) or hypoxia (H) (8% O₂) for 2 h followed by treatment with RDEA119 MEK inhibitor, then washed. SSRBC adhesion to microvascular endothelial cells was tested in intermittent flow condition assays, and results are presented as % adherent SSRBCs at a shear stress of 1-2 dynes/cm². RDEA119 inhibited the effect of Hypoxia on sickle cell adhesion. *: p<0.05 compared to sham-treated sickle cells: **: p<0.001 compared to sickle cells exposed to hypoxia. Error bars show SEM of 3 different experiments.

FIG. 3 is a set of graphs showing that epinephrine increases sickle RBC adhesion to endothelial cells via ERK activation. Sickle RBC adhesion to non-activated HUVECs was tested in intermittent flow conditions, and results are presented as % adherent sickle RBCs at a shear stress of 2 dynes/cm². Sickle RBCs (n=5 for FIG. 3A and n=3 for FIG. B) were sham-treated, or treated with 20 nM epinephrine (epi) (both FIG. 3A and FIG. 3B) or U0126 at 10 μM (FIG. 3B). Epi variably increased sickle RBC adhesion to non-treated HUVECs (FIG. 3A and FIG. 3B), and this effect was blocked with U0126 (FIG. 3B). *: p<0.0001 and **: p<0.0001 compared to sham-treated and epi-treated sickle RBCs, respectively. Error bars show SEM of 5 different experiments for FIG. 3A and 3 different experiments for FIG. 3B.

FIG. 4 is a set of graphs showing that sickle RBCs differ in their ability to activate neutrophil adhesion via ERK activation. Sickle RBCs were sham-treated, treated with epinephrine (Epi), or with 10 μM MEK inhibitor U0126, followed by treatment with Epi, and then washed. Native neutrophils (PMNs) from healthy donors were then co-incubated with treated-sickle RBCs, and assayed for their ability to adhere to HUVECs. FIG. 4A shows that PMNs adhered to some degree to ECs. Sham-treated sickle RBCs up-regulated PMN adhesion, and such increase varied among individuals (n=22; p=0.0245). FIG. 4B shows that adhesion of PMNs was much higher and also varied when cells were co-incubated with Epi-treated sickle RBCs (n-=22; p<0.0001 compared to PMNs alone). FIG. 4C shows that addition of a MEK inhibitor reduced the effect of epi-stimulated sickle RBCs on PMN adhesion compared to adhesion of epi-activated sickle RBC-mediated PMN adhesion. *:p<0.0001 compared to native PMNs; and **:p<0.0001 compared to PMN adhesion mediated by epi-treated sickle RBCs. Error bars show SEM of 4 different experiments.

FIG. 5 is a set of photographs and graphs showing that ERK in sickle RBCs promotes vaso-occlusion in nude mice in vivo. FIG. 5A, FIG. 5B and FIG. 5C are a set of photographs showing nude mice implanted with dorsal skin-fold window chambers after injection with murine TNFα. Four hours later, intravital microscopic observations of post-capillary venules and arterioles using 10× and 20× magnifications were conducted through the window chamber immediately after infusion of fluorescently labeled human sickle RBCs (n=5) that were sham-treated (FIG. 5A; panels 1, 2, 3 and 4) or treated with the MEK inhibitor RDEA119 (FIG. 5B panels 1, 2 and 3), or human normal (AA) RBCs (n=3) sham-treated (FIG. 5C; two panels on the left) or RDEA119-treated (FIG. 5C; 2 panels on the right). Vessels without adherent cells appear gray, due to the rapidly moving of fluorescence labeled RBCs. Adhesion of human sickle RBCs in enflamed vessels and vaso-occlusion are indicated with arrows and appear as white spots in the vessels. Sham-treated human sickle RBCs showed marked adhesion with intermittent vaso-occlusion (FIG. 5A), whereas RDEA119-treated sickle RBCs showed little adhesion to enflamed vessel walls (FIG. 5B). Sham-treated and RDEA119-treated human AARBCs showed no adhesion to venule walls (FIG. 5C). Scale bar=50 μm. FIG. 5D is a graph showing quantification of the fluorescence intensity of video frames of vessel and arteriole segments used to quantify adhesion in venules and arterioles of animals occupied by sickle RBCs (n=5 for each treatment). Adhesion of fluorescently labeled sham-treated sickle RBCs (sham-treated SS) and RDEA119-treated sickle RBCs (RDEA119-treated SS) in all vessels and arterioles recorded presented as fluorescence intensity [fluorescence unit (FU)] of adherent sickle RBCs. FIG. 5E-FIG. 5G are graphs showing the values of at least 180 segments of vessels and arterioles analyzed and averaged among groups of animals (n=5) to represent percentage of vessels occupied by adherent sickle RBCs (FIG. 5E); percentage of vessels with normal blood flow, slow blood flow and no blood flow (FIG. 5F), and percentage of normal flowing vessels (FIG. 5G). Error bars show SEM of 5 different experiments for each treatment condition, *: p<0.001 compared to sham-treated sickle RBCs regardless of the vessel diameter within the ranges specified for D, E and G.

FIG. 6 is a set of graphs showing that ERK in sickle RBCs mediates sickle cell adhesion and vaso-occlusion in transgenic sickle cell mice in vivo. Anesthetized sickle mice with dorsal skin-fold window chamber implants were infused with 0.025, 0.05 or 0.1 mg/kg MEK inhibitor RDEA119 or vehicle (0.02% DMSO in saline) 120 min after TNFα challenge (time 0), a time at which RBCs adhered and a vaso-occlusive crisis is established. Ten minutes following drug administration, images of the sub-dermal vasculature were recorded for 80 min between the time points of 130 and 210 min (T₁₃₀→T₂₁₀). Video frames of vessel and arteriole segments of animals were used to quantify adhesion of fluorescence-labeled murine RBCs (FU), and percentage of occluded vessels. FIG. 6A is a graph showing that murine sickle RBCs adhered markedly in vehicle-treated animals. In contrast, 0.025, 0.05 and 0.1 mg/kg RDEA119 reversed murine sickle RBC adhesion. *: p<0.0001 compared to vehicle-treated animals regardless of the vessel diameter. FIG. 6B is a graph showing adhesion of murine RBCs (FU) presented as a function of time. RDEA119 at the lowest dose (0.025 mg/kg) was able to reverse marine sickle RBCs adhesion within the first 10 min of drug administration compared to vehicle, and such effect was sustained over time. *: p<0.01 compared to vehicle-treated animals. FIG. 6C is a graph showing the percentage occluded vessels. Treatment of sickle mice with RDEA119 restored blood flow in vessels. *: p<0.05 compared to vehicle-treated animals regardless of the vessel diameter. Error bars show SEM of 4 different experiments for each treatment group.

FIG. 7 is a set of graphs showing that ERK in leukocytes is involved in leukocyte adhesion in transgenic sickle cell mice in vivo. Anesthetized sickle mice with dorsal skin-fold window chamber implants were infused with 0.025, 0.05 or 0.1 mg/kg MEK inhibitor RDEA 119 or vehicle (0.02% DMSO in saline) 120 min after TNFα challenge (time 0), a time at which leukocytes adhered and a vaso-occlusive crisis is established. Ten minutes following drug administration, images of the sub-dermal vasculature were recorded for 80 min between the time points of 130 and 210 min (T₁₃₀→T₂₁₀). Video frames of vessel and arteriole segments of animals were used to quantify adhesion of fluorescence-labeled murine leukocytes (FU). FIG. 7A is a graph showing that leukocytes adhered to enflamed venules in vehicle-treated sickle mice. However, 0.025, 0.05 and 0.1 mg/kg RDEA119 reversed leukocyte adhesion, *: p<0.0001 compared to vehicle-treated animals regardless of the vessel diameter. FIG. 7B is a graph showing adhesion of leukocytes (FU) presented as a function of time. Leukocyte adhesion was abrogated within the first 10 min of 0 025, 0.05 and 0.1 mg/kg RDEA119 administration compared to vehicle treatment, and adhesion further decreased thereafter. *: p<0.05 compared to vehicle-treated animals. Error bars show SEM of 4 different experiments for each treatment group.

FIG. 8 is a set of graphs showing that ERK activity positively correlates with sickle RBC adhesion to endothelial cells. FIG. 8A is a graph showing basal/inducible phosphorylation of ERK in sickle RBCs is variable among SCD patients. Sickle RBCs were sham-treated (unstimulated) and treated with 20 nM epinephrine (stimulated). Western blots were stained with antibodies against ERK and phosphoERK. Quantitative analysis of the data normalized according to total ERK, is presented as ERK phosphorylation intensity. Sickle RBC ERK is phosphorylated at baseline, and ERK phosphorylation is increased by epinephrine. p<0.05 compared to unstimulated cells. FIG. 8B is a graph showing relation of ERK activity level to sickle RBC adherence to ECs. ERK activity positively relates to sickle RBC adherence (n=8, r²⁼0.74, p<0.05, correlation coefficient=0.86).

FIG. 9 is a set of figures showing that activation of Gas protein increased SSRBC adhesion to endothelial cells via activation of tyrosine kinases. FIG. 9A is a schematic depiction of GPCR pathways along with inhibitors and stimuli of the kinases of these pathways regulating SSRBC adhesion. FIGS. 9B-E are a set of graphs showing adherence of RBCs which were sham-treated, or treated with Pertussis toxin (PTx) (0.5, 1 or 2 μg/ml) (FIGS. 9B-D), 1 μg/ml Cholera toxin (CTx) (FIG. 9B) or phenylarsine oxide (0.1, 10, 20, 40 and 80 μM) (FIG. 9E), or pretreated with 1 μM PP1, 1 μM PP2 or 1 μM piceatannol prior to treatment with 1 μg/ml PTx (FIG. 9C and FIG. 9D). Treated RBCs were tested for adhesion to HUVECs in intermittent flow conditions at different shear stresses. Data are presented as % adherent RBCs (FIG. 9B) or SSRBCs (FIG. 9C-FIG. 9E) at a shear stress of 2 dynes/cm². Each graph is representative of 3 different experiments. Error bars show standard error of the mean (SEM). *: p<0.001 compared to sham-treated RBCs; **: p<0.01 compared to PTx-treated RBCs.

FIG. 10 is a set of figures showing inhibition of Gai protein increased phosphorylation of p72^{syk} and p60-c-Src tyrosine kinases in intact SSRBCs. FIG. 10A-FIG. 10G show the kinase expression results for twenty μg of membrane protein ghosts (SSRBC ghosts, n=7, SS1, SS2, SS3, SS4, SS5, SS6 and SS7; and normal RBC ghosts, n=7, AA1, AA2, AA3AA4, AA5, AA6 and AA7) were used per lane. FIG. 10A-FIG. 10D show Western blots and corresponding graphs showing the kinase expression results of protein ghosts which were stained with antibodies against phosphorylated p60-c-Src [p-p60-c-Src (SS1, SS2, SS3, SS4; and AA1, AA2, AA3, AA4)] and p60-c-Src [p60-c-Src (SS1, SS2, SS3, SS4, SS5, SS6, SS7, and AA1, AA2, AA3, AA4, AA5, AA6, AA7)]; and phosphorylated p72^{syk} [p-Syk (SS1, SS2, SS3, SS4; and AA1, AA2, AA3, AA4)] and p72^{syk}[Syk (SS1, SS2, SS3, SS4, SS5, SS6, SS7; and AA1, AA2, AA3, AA4, AA5, AA6, AA7)]. Antibody against glycophorin C was used as a loading control. FIG. 10C and FIG. 10D show the quantitative analysis of the data presented as relative kinase expression [SSRBCs (n=7) and AARBCs (n=7)], and as relative kinase phosphorylation [SSRBCs (n=4) and AARBCs (n=4)]. Error bars show SEM. *: p<0.001 (for kinase expression) and p<0.01 (for kinase phosphorylation) compared to normal cells. FIG. 10E-FIG. 10G are Western blots and corresponding graphs showing SSRBCs (FIG. 10.E FIG. 10F and FIG. 10G) and AARBCs (FIG. 10F and FIG. 10G) were sham-treated, or treated with 1 μg/ml PTx or 1 μM PP1, or pretreated with 1 μM PP1, 1 μM PP2 or 1 μM piceatannol prior to treatment with 1 μg/ml PTx. Western blots of protein ghosts were stained with antibodies against p-p60-c-Src, p60-c-Src, p-Syk and Syk. Antibody against glycophorin C was used as a loading control. Quantitative analysis of the data (normalized according to total kinase expression) is presented as fold change in p60-c-Src phosphorylation (FIG. 10E and FIG. 10F) and Syk phosphorylation (FIG. 10G). Error bars show SEM of 3 different experiments (n=3) for each FIG. 10E, FIG. 10F and FIG. 10G. *: p<0.001 compared to sham-treated cells; **: p<0.01 compared to PTx-treated SSRBCs; ***: p<0.00 compared to sham-treated AARBCs.

FIG. 11 is a graph showing the number of hospitalizations for painful episodes in the past 12 months. 165 patients with SCD were hospitalized in the past 12 months. Of these 165 patients, the percentages of the patients hospitalized for zero pain crises, one pain crisis, between 2 and 4 pain crises, and for more than 4 pain crises are presented.

### DETAILED DESCRIPTION

Effective therapies are desperately needed in sickle cell disease (SCD) to prevent and curtail the recurrent painful vaso-occlusive crises that lead to multi-organ damage, an inevitable consequence of this disease. Current treatments for SCD achieve only symptomatic relief and have no demonstrated efficacy in preventing organ damage. Therapies that focus on ameliorating sickle red blood cell (RBC) dehydration, interfering with chemical-physical processes during erythrocyte-endothelial adhesion events, or targeting RBC adhesion molecules, to prevent RBC-endothelial cell interactions have shown little to no therapeutic benefit. While it is known that the abnormal sickle cell adhesion is the proximate cause of events that precipitate vaso-occlusion, there has been no attempt to target the signaling mechanisms required for sickle cell adhesion. The current major limitation in developing therapeutics for vaso-occlusive crises is our poor understanding of the specific signaling mechanisms that lead to increased sickle cell adhesion to endothelium, the subsequent stimulation of leukocyte adhesion, and the formation of vaso-occlusive cell aggregates. An in-depth understanding of sickle cell signaling pathways that mediate adhesion at both the biochemical and physiological levels will be required to successfully exploit these pathways for therapeutic and diagnostic purposes and to develop efficacious pathway-selective drugs with minimal side effects.

Earlier the present inventor suggested that the mitogen-activated protein kinase MAPK)/the extracellular signal-regulated kinase 1/2 (ERK) is present at higher abundance in sickle RBCs than in normal RBCs and is bound to the cytoplasmic membrane³⁷ . The inventor has shown that ERK is active in enucleated sickle RBCs, and that triggering this kinase promotes activation of signaling pathways and consequent RBC adhesion to the endothelium.³⁷Stimulation of β₂-adrenergic receptors (β₂ARs) on sickle RBCs by epinephrine for a brief period of time increases activation of the ERK signaling cascade, which is involved in phosphorylation of the RBC adhesion receptor ICAM-4. It was also found that the ERK consensus motifs on dematin and α- and β-adducins undergo increased serine phosphorylation, indicating that these cytoskeletal proteins are substrates for ERK.

ERK has been implicated in EPO-induced erythroid cell proliferation and survival,³⁸ and the present inventor has now demonstrated that the activity of this kinase and its upstream signal are conserved in mature sickle RBCs, and can be increased by either epinephrine or EPO treatment. In some instances, ERK is hyperactive without stimulation of sickle RBCs, and increased activation of this kinase can increase within 1 minute of sickle RBC exposure to epinephrine. In contrast, in normal RBCs, despite the abundance of ERK, ERK is not, active at baseline and fails to become phosphorylated/activated with epinephrine or forskolin stimulation. See International Application Publication No, WO 2012/149547, which is incorporated herein by reference in its entirety. The inability of ERK to undergo activation in normal RBCs suggests that the activity of ERK itself and/or at least one of the upstream effectors required for ERK activation is lost. Indeed, investigators have previously described that RBCs undergo maturation-related loss of multiple protein kinase activities, including PKA, PKC, and casein kinases.³⁹ . In contrast, although sickle RBCs are also fully differentiated, but younger in age than normal RBCs since sickle RBCs enter the circulation while they are not completely mature and are removed from the circulation sooner than normal RBCs, the present inventor has found that preservation of ERK activity and its downstream signaling molecules appears to be involved at least in the abnormal activation of sickle RBC adhesive function.

Our data further implicate involvement of the protein Gₛ and cAM P/PKA pathways as upstream mediators in activation of ERK and its downstream signal transduction pathway. Our findings are consistent with studies by Schmitt and Stork²⁰ demonstrating that isoproterenol stimulation of endogenous β₂ARs activated ERK in HEK293 cells via a cAMP-dependent PKA pathway, and ERK activation increased by treatment with PTx, which inactivates the protein Gaᵢ. In addition to PKA, the inventor has also identified a role for the tyrosine kinase p72^{Syk} in activation of ERK in sickle RBCs, while excluding involvement of p56^{lck} related Src family tyrosine kinases. Thus, in sickle RBCs, PKA and the tyrosine kinase p72^{Syk} are implicated in ERK activation, acting most likely in concert to regulate the MEK/ERK signaling pathway.

The engagement of epinephrine in regulation of sickle RBC adhesion to the endothelium suggests that the MEK/ERK signal can promote an adhesive, vaso-occlusive pathology. Epinephrine-induced adhesion of sickle RBCs to non-activated endothelial cells requires ICAM-4 phosphorylation, which occurs via the cAMP/PKA/MEK/ERK signaling pathway. Furthermore, the adhesive function of sickle RBCs appeared to be related to the extent of ERK phosphorylation/activation, since both increased or decreased similarly depending on the time of cell exposure to epinephrine. Additionally, basal cAMP levels, the upstream effector of MEK/ERK, were much higher in sickle RBCs than in normal cells, suggesting that the increased level of cAMP in sickle RBCs reflects at least in part the persistence of the abnormal ERK activation and RBC adhesive phenotype. However, although epinephrine increased cAMP levels in only 50% of the SCD patient samples tested, cAMP production, which seems to be needed to activate ERK signaling in these sickle cells, was also influenced by the duration of cell exposure to epinephrine. This may be explained at least in part by the dramatic decrease in the abundance of phosphopeptides within CAP1 in sickle RBCs due to continued cell exposure to epinephrine stimulation. PKA might also exert a negative feedback loop through activation of phosphodiesterases, resulting in cAMP hydrolysis switching off downstream signaling because of the extended cell exposure to epinephrine. CAPs are not only involved in adenylate cyclase (AC) association, but in actin binding, SH3 binding, and cell morphology maintenance as well. Previous observations of increased normal RBC membrane filterability after epinephrine treatment for 20 min, explain the enhanced phosphorylated CAP1 in normal RBCs after 30 min epinephrine exposure. Furthermore, Shain et al.⁴⁴ have suggested that maintenance of altered cell morphology required persistent increased cAMP levels due to continuous βAR stimulation.

In contrast, our data suggest that when an increase in ERK activation occurs within 1 min of cell exposure to epinephrine, persistent β₂AR stimulation has a negative effect on ERK activation and consequently the RBC adhesive function. Based on this analysis, it is expected that inhibition of b-Raf or c-Raf will result in similar effects in sickle RBCs as these are additional upstream. activators in this pathway.

ERK signaling activation was also involved in adhesion of leukocytes and vaso-occlusion triggered by inflammation in a sickle mouse animal model of acute vaso-occlusive crises. Leukocyte recruitment and adhesion to activated-endothelial cells are an extremely dynamic process in which most adherent leukocytes remain adherent, some continuously crawl along the venular endothelium while others detach to return in the circulation. Extravasated leukocytes account for a relatively minor subset of leukocytes that have adhered. Long after the inflammatory challenge was initiated and occurrence of vasoocclusion, inhibition of ERK with the MEK inhibitor RDEA119 efficiently reduced the number of adherent leukocytes and restored blood flow. Thus, MEK-dependent ERK activation it leukocytes appears to also play a crucial role in their recruitment and adherence to the vascular endothelium arid initiation of vasoocclusion sickle mice in vivo.

The inventor believes that key components associated with the ERK pathway could prove to be potential therapeutic targets to alleviate symptoms associated with a hemoglobinopathy such as sickle cell disease. The inventor has further demonstrated that G protein-coupled receptor kinase 2 (GRK2) and β-arrestin1/2 could either be triggered by activation of the mitogen activated protein kinase ERK pathway or act downstream of ERK. Thus inhibitors of these proteins may result in alleviation of symptoms associated with a hemoglobinopathy such as sickle cell disease. Increased membrane translocation of GRK2 and β-arrestin1/2 and GRK2 and β-arrestin1 activation in sickle RBCs, and activation of MEK/ERK signaling in leukocytes may therefore be associated with the pathophysiology of sickle cell disease, making this pathway a therapeutic target for preventing and treating vaso-occlusion, and reversing established vaso-occlusion. Thus these inhibitors of these pathways provide methods of alleviating the symptoms of hemoglobinopathies, such as sickle cell disease and β-thalassemia. These methods involve administering ERK, Ras, BRAF/Raf1 MEK, GRK2, Syk, p60-c-Src, a tyrosine kinase the inventor has shown to be also activated via the Gs protein to mediate sickle cell adhesion, and/or β-arrestin1/2 inhibitors. Furthermore, sickle RBCs are characterized by a panopoly of abnormalities, including polymerization of deoxygenated HbS, persistent oxidative membrane damage associated with HbS cyclic polymerization, abnormal activation of membrane cation transports, cell dehydration, and cytoskeletal dysfunction. Thus, ERK, Ras, BRAF/Raf1 MEK, GRK2, Syk, p60-c-Src and/or β-arrestin1/2 inhibition may result not only in amelioration of vaso-occlusion, but also other symptoms of sickle cell disease.

As clinical care has improved for patients with hemoglobinopathies and the patient population is aging due to improved survival, new issues are evolving. In addition to complications due to hemolysis, these new issues include long-term complications of infection with hepatitis C (HCV), thrombosis, and fertility. Increasing evidence of thrombotic risk in patients with SCD, and with thalassemia intermedia and thalassemia major is being reported in the literature. Further, the improved lifespan and clinical status of the affected population has allowed preservation of fertility and successful term pregnancies in some patients. Indeed, in a recent review, compelling clinical evidence for increased risk of thrombosis in patients with not only β-thalassemia intermedia, but also β-thalassemia major, α-thalassemia syndromes and hemoglobin E/β-thalassemia (E/β-thal) was presented. Therefore, it is critical to determine clinical risk, which will help develop preventive care and treatment plans for these patients.

Biologic risk factors for thrombosis include splenectomy, red cell phosphatidylserine exposure, and plasma coagulation factor abnormalities. Comparison of E/β-that patients with splenectomy, without splenectomy and age-matched controls demonstrated statistically increased levels of thrombin-antithrombin III (“TAT”) complex in the splenectomized patients compared to the other two groups. Levels of prothrombin fragment 1.2 and RBC phosphatidylserine were statistically increased in the splenectomized patients when compared to the controls. These findings suggest ongoing thrombin generation related to anionic phospholipid exposure. Plasma β₂-thromboglobulin and platelet aggregation studies demonstrated statistically significant hyperaggregation in the splenectomized group when compared to the nonsplenectomized and normal controls. Increased phosphotidylserine exposure by red cells is known to contribute to formation of red cell aggregates and adhesion of the red cell to the endothelium in sickle cell disease. Therefore, it is also possible that increased phosphatidylserine in red cells in thalassemia patients may participate in increased cell aggregate formation blocking small blood vessels in these patients.

To predict clinical vascular complications associated with sickle cell disease and thalassemia, new objective biomarkers are needed to assess and guide clinical intervention and to speed up the development of new therapies. Endothelial damage and activation from sickle RBC-endothelial interactions leading to up-regulation of gene expression of endothelial adhesion molecules VCAM-1, E-selectin and ICAM-1 may contribute to vascular complications. Studies have found increased soluble VCAM1 (sVCAM-1) expression correlated with a clinical manifestation of endothelial dysfunction and was associated with the risk of mortality in a cohort of SCD patients in a steady state. Sickle RBCs have been reported to induce endothelial adhesion molecules and endothelial damage. In addition, studies have found a relationship between clinical manifestations and expression of adhesion molecules on leukocytes. Also, leukocytosis, in the absence of infection, is common in SCD patients and correlates well with clinical severity. Our findings provided in the Examples suggest that in SCD in particular, ERK signaling activation in, both sickle RBCs and leukocytes could affect adhesion of sickle RBCs and leukocytes, sickle RBC-induced activation of neutrophils and neutrophil adhesion, and subsequently, vascular endothelial injury and mortality. The ability to predict clinical'vascular complications, provide a diagnosis thereof and provide a clinical treatment plan or administer pharmaceutical agents to reverse or stop progression of these events is described herein.

### Definitions

The subject matter disclosed herein is described using several definitions, as set forth below and throughout the application.

Unless otherwise noted, the terms used herein are to be understood according to conventional usage by those of ordinary skill in the relevant art. In addition to the definitions of terms provided below, it is to be understood that as used in the specification, embodiments, and in the claims, “a”, “an”, and “the” can mean one or more, depending upon the context in which it is used.

As used herein, “about,” “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” or “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.”

As used herein, the terms “patient” and “subject” may be used interchangeably and refer to one who receives medical care, attention or treatment. As used herein, the term is meant to encompass a person diagnosed with a disease such as a hemoglobinopathy or at risk for developing a hemoglobinopathy (e.g., a person who may be genetically homozygous or heterozygous for a sickle cell-causing mutation, but is not symptomatic). A “patient in need thereof” may include a patient having, suspected of having, or at risk for developing a hemoglobinopathy or symptoms thereof. The subjects may be humans.

As used herein, the term “treatment,” “treating,” or “treat” refers to care by procedures or application that are intended to alleviate symptoms of a disease (including reducing the occurrence of symptoms of the disease). Although it is preferred that treating a condition or disease such as a hemoglobinopathy will result in an improvement of the condition, the term treating as used, herein does not indicate, imply, or require that the procedures or applications are at all successful in alleviating symptoms associated with any particular condition. Treating a patient may result in adverse side effects or even a worsening of the condition, which the treatment was intended to improve. Treating may include treating a patient having, suspected of having, or at risk for developing a hemoglobinopathy or symptoms thereof.

Cells may be contacted with the agent directly or indirectly in vivo, in vitro, or ex vivo. Contacting encompasses administration to a cell, tissue, mammal, patient, or human. Further, contacting a cell includes adding an agent to a cell culture. Other suitable methods may include introducing or administering an agent to a cell, tissue, mammal, or patient using appropriate procedures and routes of administration as defined above.

As used herein the term “effective amount” refers to the amount or dose of the agent, upon single or multiple dose administration to the subject, given acutely or chronically, which provides the desired effect in the subject under diagnosis or treatment. The disclosed methods may include administering an effective amount of the disclosed agents (e.g., as present in a pharmaceutical composition) for treating a hemoglobinopathy in the patient, whereby the effective amount alleviates symptoms of the hemoglobinopathy (including reducing the occurrence of symptoms of the hemoglobinopathy).

An effective amount can be readily determined by the attending diagnostician, as one skilled in the art, by the use of known techniques and by observing results obtained under analogous circumstances. In determining the effective amount or dose of agent administered, a number of factors can be considered by the attending diagnostician, such as: the species of the patient; its size, age, and general health; the particular symptoms or the severity of the hemoglobinopathy; the response of the individual patient; the particular agent administered; the mode of administration; the bioavailability characteristics of the preparation administered; the dose regimen selected; the use of concomitant medication; the length of use of the concomitant medication and other relevant circumstances.

The phrase “alleviates at least one symptom,” as used herein, means that a particular treatment results in a lessening of at least one symptom of a disease. Such lessening of a symptom may be a qualitative or quantitative reduction in the severity of the symptom, or may be a reduction in the number of occurrences of the symptom; even though each occurrence may be as severe as it was before the treatment (one or more occurrences may also be less severe). Non-limiting exemplary symptoms of sickle cell disease include vaso-occlusion, acute and chronic painful episodes, chronic hemolysis (aplastic crises), avascular necrosis, infection, end-organ damage, acute chest syndrome, kidney damage, stroke, leg ulceration, priapism, and decreased life expectancy. Non-limiting exemplary symptoms of thalassemia include hemolysis, erythroid hyperplasia, biliary tract disease, infection, leg ulcers, extramedullary hematopoiesis, increased risk for developing thromboembolic phenomena or cell aggregates, liver, kidney and heart damage, and decreased life expectancy.

The term “hemoglobinopathy,” as used herein, refers to a condition that is caused by a genetic mutation in a globin gene that results in a mutated hemoglobin α chain or β chain protein, or a condition that is caused by a genetic mutation that results in an abnormal ratio of hemoglobin α chain to β chain or crossover fusion products of 2 globin genes. Non-limiting exemplary hemoglobinopathies include sickle cell disease (including, but not limited to, homozygous for hemoglobin S and a variety of sickle cell syndromes that result from inheritance of the sickle cell gene in compound heterozygosity with other mutant beta globin genes, including, but not limited to, hemoglobin SC disease (HbSC), sickle beta(+) thalassemia, sickle beta(0) thalassemia, sickle alpha thalassemia, sickle delta beta(0) thalassemia, sickle Hb Lepore, sickle HbD, sickle HbO Arab, and sickle HbE),β-thalassemia (including, but not limited to, β-thalassemia major (also known as Cooley's anemia) andβ-thalassemia intermedia, and hemoglobin H disease (α-thalassemia with α⁺-α⁰ phenotype)).

Non-limiting exemplary genetic mutations that cause sickle cell disease include Hb SS, which is hemoglobin with an E6V mutation in each of the two hemoglobin β chains; Hb SS, which is hemoglobin with one β chain with an E6V mutation and one β chain with an E6K mutation; Hb SD, which is hemoglobin with one β chain with an E6V mutation and one β chain with a β1.21 Glu-> Gln mutation; sickle-HbO Arab, which is hemoglobin with one β chain with an E6V mutation and one β chain with a β121(GH4)gGlu->Lys mutation; and Hb SE, which is hemoglobin with one β chain with an E6V mutation and one β chain with an E26K mutation.

Non-limiting exemplary genetic mutations that cause β-thalassemia include various β-mutations, such as IVS II-I, CD36/37, CD41/42, CD39; IVS1-6; IVS1-110, CD71/72, IVS1-5, IVS1-1, CD26, IVS2-654, CAP+1, CD19, -28, -29, IVS1-2, InCD (T-G) and CD17; and rare β-mutations, i.e. InCD (A-C), CD8/9, CD43, -86, CD15, Poly A, Poly T/C, IVS2-1, CD1, CD35/36, CD27/28, CD16, CD37, and 619bpDEL.

Non-limiting exemplary genetic mutations that cause Hb H disease include α⁺-α⁰ phenotypes such as α2 Poly A (AATAAA→AATA--), α2 Poly A (AATAAA→AATGAA), and α2 Poly A (AATAAA→AATAAG); α⁺ phenotypes such as α2 CD 142 (TAA→CAA), α2 CD 142 (TAA→AAA), and α2 CD 142 (TAA→TAT); and α⁰ phenotypes such as —α^{3.7} Init CD (ATG→GTG), --^{SEA}, --^{THAI}, --^{MED II}, --^{BRIT}, --^{MED I}, --^{SA}, --(α)^{20.5}, and --^{FIL}.

The term “MEK inhibitor,” as used herein, refers to an inhibitor of MEK kinase activity. A MEK inhibitor may be any type of molecule, including, but not limited to, small molecules and expression modulators (such as antisense molecules, microRNAs, siRNAs, etc.), and may act directly on the MEK protein, may interfere with expression of the MEK protein (e.g., transcription, splicing, translation, and/or post-translational processing), and/or may prevent proper intracellular localization of the MEK protein. Exemplary MEK inhibitors include, but are not limited to, U0126, PD98059, PD-334581, GDC-0973, CIP-137401, ARRY-162, ARRY-300, PD318088, PD0325901, CI-1040, BMS 777607, AZD8330, AZD6244, RDEA119, GSK1120212 and AS703026.

The term “ERK inhibitor,” as used herein, refers to an inhibitor of ERK kinase activity. An ERK inhibitor may be any type of molecule, including, but not limited to, small molecules and expression modulators (such as antisense molecules, microRNAs, siRNAs, etc.), and may act directly on the ERK protein, may interfere with expression of the ERK protein (e.g., transcription, splicing, translation, and/or post-translational processing), and/or may prevent proper intracellular localization of the ERK protein. A nonlimiting exemplary ERK inhibitor is AEZS-131.

The term “Raf inhibitor,” as used herein, refers to an inhibitor of b-Raf kinase activity and/or c-Raf kinase activity. A Raf inhibitor may be any type of molecule, including, but not limited to, small molecules and expression modulators (such as antisense molecules, microRNAs, siRNAs, etc.), and may act directly on the Raf protein, may interfere with expression of the Raf protein (e.g., transcription, splicing, translation, and/or post-translational processing), and/or may prevent proper intracellular localization of the Raf protein. Nonlimiting exemplary Raf inhibitors include sorafenib tosylate, GDC-0879, PLX-4720, regorafenib, PLX-4032, SB-590885-R, RAF265, GW5074, XL281, and GSK2118436.

A table providing additional information on some of the exemplified MEK, ERK, and Raf inhibitors is provided below as Table 1.

TABLE 1
Non-limiting exemplary inhibitors of MEK, ERK, and/or B-Raf

| Inhibitor | Alternate name(s) | Structure or source |
|---|---|---|
| U0126 | U0126-EtOH | 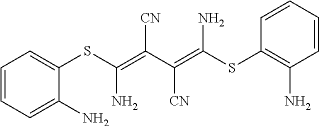 [SMILES: N#CC(=C(/N)Sc1ccccc1N)/C(C#N)=C(\N)Sc1ccccc1N] |
| | | 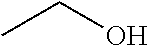 |
| PD98059 | | 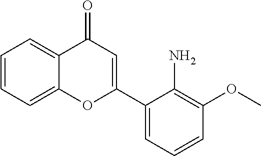 [SMILES: COc1cccc(-c2cc(=O)c3ccccc3o2)c1N] |
| PD-334581 | | 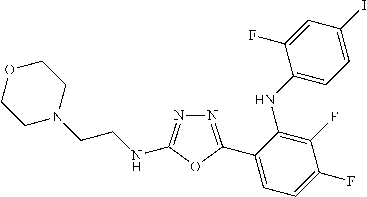 [SMILES: Fc1cc(I)ccc1Nc1c(-c2nnc(NCCN3CCOCC3)o2)ccc(F)c1F] |
| | | Chemical Formula: C₂₀H₁₉F₃IN₅O₂ |
| | | Molecular Weight: 545.30 |
| GDC-973 | XL518 | Genentech |
| CIP-137401 | CIP-1374 | Allostem Therapeutics |
| ARRY-162 | | Array BioPharma/Novartis |
| ARRY-300 | | Array BioPharma/Novartis |
| PD0318088 | | 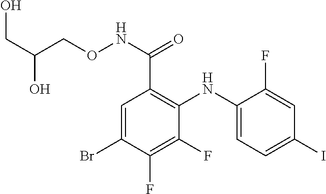 [SMILES: O=C(NOCC(O)CO)c1cc(Br)c(F)c(F)c1Nc1ccc(I)cc1F] |
| PD0325901 | | 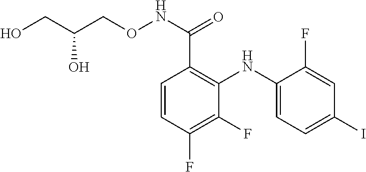 [SMILES: O=C(NOC[C@H](O)CO)c1ccc(F)c(F)c1Nc1ccc(I)cc1F] |
| CI-1040 | PD184352 | 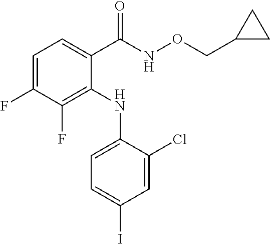 [SMILES: O=C(NOCC1CC1)c1ccc(F)c(F)c1Nc1ccc(I)cc1Cl] |
| BMS 777607 | | 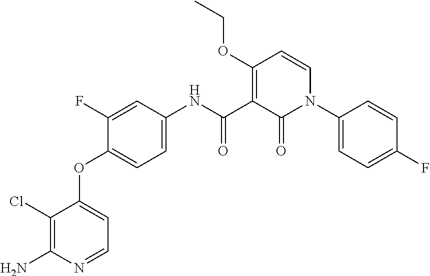 [SMILES: CCOc1ccn(-c2ccc(F)cc2)c(=O)c1C(=O)Nc1ccc(Oc2ccnc(N)c2Cl)c(F)c1] |
| AZD8330 | ARRY-424704 ARRY-704 | 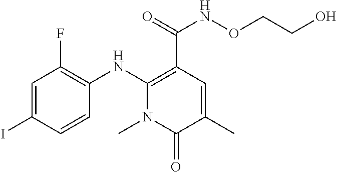 [SMILES: Cc1cc(C(=O)NOCCO)c(Nc2ccc(I)cc2F)n(C)c1=O] |
| AZD 6244 | Selumetinib | 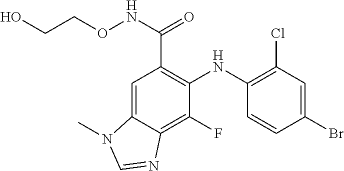 [SMILES: Cn1cnc2c(F)c(Nc3ccc(Br)cc3Cl)c(C(=O)NOCCO)cc21] |
| AS703026 | MSC1936369B | 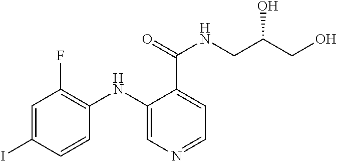 [SMILES: O=C(NC[C@H](O)CO)c1ccncc1Nc1ccc(I)cc1F] |
| AEZS-131 | | Aeterna Zentaris Inc. |
| sorafenib tosylate | BAY 43-9006 AZ 628 | 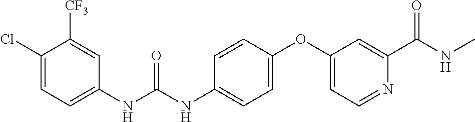 [SMILES: CNC(=O)c1cc(Oc2ccc(NC(=O)Nc3ccc(Cl)c(C(F)(F)F)c3)cc2)ccn1] |
| | | C₇H₈O₃S |
| GDC-0879 | | 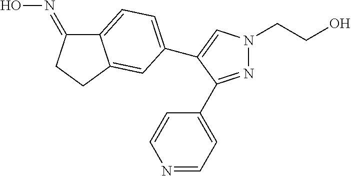 [SMILES: OCCn1cc(-c2ccc3c(c2)CC/C3=N\O)c(-c2ccncc2)n1] |
| PLX-4720 | | 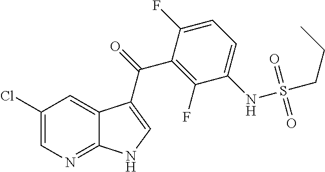 [SMILES: CCCS(=O)(=O)Nc1ccc(F)c(C(=O)c2c[nH]c3ncc(Cl)cc23)c1F] |
| regorafenib | BAY 73-4506 | 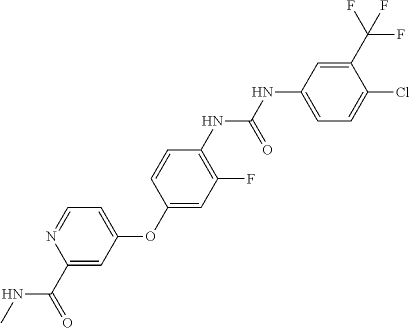 [SMILES: CNC(=O)c1cc(Oc2ccc(NC(=O)Nc3ccc(Cl)c(C(F)(F)F)c3)c(F)c2)ccn1] |
| PLX-4032 | RG7204 | 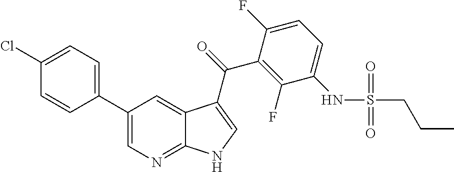 [SMILES: CCCS(=O)(=O)Nc1ccc(F)c(C(=O)c2c[nH]c3ncc(-c4ccc(Cl)cc4)cc23)c1F] |
| SB-590885 | | 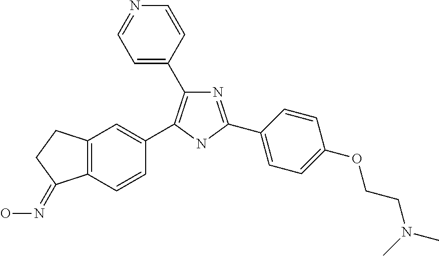 [SMILES: CN(C)CCOc1ccc(-c2nc(-c3ccncc3)c(-c3ccc4c(c3)CC/C4=N\O)[nH]2)cc1] |
| RAF265 | CHIR-265 | 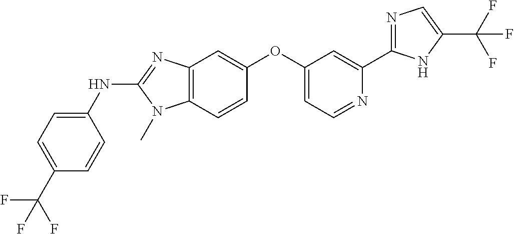 |
| GW5074 | | 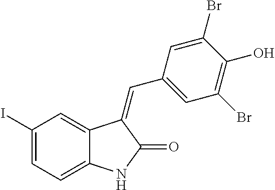 [SMILES: O=C1Nc2ccc(I)cc2/C1=C/c1cc(Br)c(O)c(Br)c1] |
| XL281 | BMS-908662 | Exelixis |
| GSK2118436 | | GlaxoSmithKline |

The term “β-arrestin1/2 inhibitor,” as used herein, refers to an inhibitor of β-arrestin1/2 kinase membrane translocation and activity. A β-arrestin1/2 inhibitor may be any type of molecule, including, but not limited to, small molecules, inhibitory antibodies and expression modulators (such as antisense molecules, microRNAs, siRNAs, aptamers, etc.), and may act directly on the β-arrestin1/2 protein, may interfere with expression of the β-arrestin1/2 protein (e.g., transcription, splicing, translation, and/or post-translational processing), and/or may prevent improper intracellular localization and/or membrane translocation, and/or phosphorylation and/or activation of the β-arrestin1/2 protein.

The term “GRK2” inhibitor, as used herein, refers to an inhibitor of GRK2 kinase membrane translocation and activity. A GRK2 inhibitor may be any type of molecule, including, but not limited to, small molecules, antibodies and expression modulators (such as antisense molecules, microRNAs, siRNAs, aptamers, etc.), and may act directly on the GRK2 protein, may interfere with expression of the GRK2 protein (e.g., transcription, splicing, translation, and/or post-translational processing), and/or may prevent improper intracellular localization and/or membrane translocation and/or phosphorylation and/or activation of the GRK2 protein. A GRK2 inhibitor includes but is not limited to βARK 1.

The term “Ras inhibitor,” as used herein, refers to an inhibitor of Ras kinase membrane translocation and activity. A Ras inhibitor may be any type of molecule, including, but not limited to, small molecules, antibodies and expression modulators (such as antisense molecules, microRNAs, siRNAs, aptamers, etc.), and may act directly on the Ras protein, may interfere with expression of the Ras protein (e.g., transcription, splicing, translation, and/or post-translational processing), and/or may prevent improper intracellular localization and/or membrane translocation, and/or phosphorylation and/or activation of the Ras protein. A Ras inhibitor includes but is not limited to farnesylthiosalicyclic acid or other farnesyltransferase inhibitors.

The term “Syk inhibitor,” as used herein, refers to an inhibitor of Syk kinase activity. A Syk inhibitor may be any type of molecule, including, but not limited to, small molecules, antibodies and expression modulators (such as antisense molecules, microRNAs, siRNAs, aptamers, etc.), and may act directly on the Syk protein, may interfere with expression of the Syk protein (e.g., transcription, splicing, translation, and/or post-translational processing), and/or may prevent improper intracellular localization and/or phosphorylation and/or activation of the Syk protein. A Syk inhibitor includes but is not limited to fostamatinib and piceatannol.

The term “p60-c-Src inhibitor,” as used herein, refers to an inhibitor of p60-c-Src kinase membrane translocation and activity. A p60-c-Src inhibitor may be any type of molecule, including, but not limited to, small molecules, antibodies and expression modulators (such as antisense molecules, microRNAs, siRNAs, aptamers, etc.), and may act directly on the p60-c-Src protein, may interfere with expression of the p60-c-Src protein (e.g., transcription, splicing, translation, and/or post-translational processing), and/or may prevent improper intracellular localization and/or membrane translocation, and/or phosphorylation and/or activation of the p60-e-Src protein. A p60-c-Src inhibitor includes but is not limited to PP1, PP2, dosatinib bosutinib, bafetinib, AZD-530, XL1-999, KX01 and KL228.

As used herein, “control level” “reference lever” or “control cells” indicates a control level of the marker(s) as found in normal (i.e. non-sickle RBC or cells from an individual with a hemoglobinopathy) or in sickle RBC cells from a subject not experiencing vascular-endothelial occlusion or other form of vascular injury. Suitably, the control level or reference level is a control level of the markers' expression of activation below which the risk of vascular-endothelial injury are low. A 1.5, 1.7, 2.0, 2.2, 2.5 or 3.0 fold increase in expression or activation of the marker above that of the controls is indicative of a high risk of vaso-occlusion or other vascular-endothelial injury.

In some embodiments, methods of alleviating at least one symptom of a hemoglobinopathy in a subject are provided. The methods include obtaining a sample including red blood cells from a subject and determining the level of expression, activation or membrane translocation of at least one marker selected from the group consisting of ERK, Ras, BRAF/Raf1, MEK,β-arrestin1/2, Syk, p60-c-Src or GRK2 in the sample and comparing the level of expression, activation or membrane translocation of the marker to a reference or control level. The level of the marker and comparison to the control or reference level allows assessment of the likelihood of a symptom of the hemoglobinopathy. In subjects with increased levels of expression, activation or membrane translocation of at least one of the markers, a treatment plan including an inhibitor of at least one of the markers can be developed. Such methods may further comprise administering to the subject an agent selected from a MEK inhibitor, an ERK inhibitor, a Raf inhibitor, aβ-arrestin1/2 inhibitor, a Ras inhibitor, a Syk inhibitor, a p60-c-Src inhibitor and a GRK2 inhibitor. The agents may be administered to subjects with elevated expression or activation of at least one of MEK, ERK, Raf, β-arrestin1/2, Ras, Syk, p60-c-Src, or GRK2. The inhibitor administered need not be directed to the same marker that is being measured. In other words, GRK2 activation could be measured, but subjects could be treated with a MEK inhibitor. The cells in the sample may be treated with at least one of cholera toxin (at 0.5-2 μg/mL for 10 min), pertussis toxin (at 0.5-2 μg/ml for 10 min), TNF-β, epinephrine or exposure to hypoxia (8% oxygen for 2 hours) prior to measuring the level of expression or activation. Methods of measuring the expression or activation of these markers are available to those of skill in the art and include but are not limited to, Western blotting, ELISA, rtPCR, Northern blotting, phosphorylation analysis, and kinase activity assays. Non-limiting exemplary hemoglobinopathies include β-thalassemia, sickle cell disease and Hemoglobin H.

For the treatment of sickle cell disease or other hemoglobinopathies, in some embodiments, at least one symptom that may be alleviated by administering the agents described herein is selected from vaso-occlusion, acute or chronic painful episodes, chronic hemolysis (aplastic crises), endothelial dysfunction, endothelial injury, avascular necrosis, infection, end-organ damage, and erythroid hyperplasia. In some embodiments, alleviating a symptom of sickle cell disease means reducing the amount, frequency, duration or severity of the symptom. For example, for vaso-occlusion, in some embodiments, alleviating the symptom includes preventing, reducing and/or reversing the average size of the vaso-occlusions, and/or reducing the number and/or frequency of vaso-occlusions. Further, alleviating a symptom may or may not result in a reduction in the discomfort experienced by the patient as a result of the symptom. That is, in some embodiments, while the number and/or average size of vaso-occlusions may he reduced following a treatment described herein, the patient may or may not experience a similar reduction in acute or chronic pain caused by vaso-occlusion.

In some embodiments, when vaso-occlusion is alleviated by administration of an agent described herein, acute painful episodes are also alleviated (i.e., the number and/or severity is reduced). In some embodiments, when vaso-occlusion is alleviated by administration of an agent described herein, hemolysis is also alleviated. In some embodiments, vascular endothelial injury is alleviated by administration of an agent described herein. In some embodiments, when hemolysis is alleviated by administration of an agent described herein, the incidence of infections is reduced. In some embodiments, when hemolysis is alleviated by administration of an agent described herein, erythroid hyperplasia is also alleviated. In some embodiments, when vaso-occlusion and/or hemolysis are alleviated by administration of an agent described herein, end-organ damage or premature death is also alleviated. Administration of an agent during as acute symptomatic episode may stop the progression of or reverse symptoms.

Vaso-occlusion may be caused by cellular adhesion in the blood vessels. The cellular adhesion may involve sickle red blood cells adhering to endothelial cells or leukocytes. The adhesion may be due to multicellular aggregates. In some embodiments, methods of inhibiting and/or reversing adhesion of sickle red blood cells to endothelial cells are provided. In some embodiments, methods of inhibiting and/or reversing adhesion of sickle red blood cells to leukocytes are provided. In some embodiments, methods of inhibiting and/or reversing activation of leukocytes and leukocyte adhesion by sickle red blood cells are provided. Such methods comprise, in some embodiments, contacting the sickle red blood cells with an agent selected from a MEK inhibitor, an ERK inhibitor, a Raf inhibitor, a Ras inhibitor, a Syk inhibitor, a p60-c-Src inhibitor, a β-arrestin1/2 inhibitor and a GRK2 inhibitor.

In some embodiments, methods of inhibiting adhesion of sickle red blood cells to endothelial cells in a patient are provided. In some embodiments, methods of inhibiting adhesion of sickle red blood cells to leukocytes and sickle red blood cell-induced leukocyte activation and adhesion to endothelial cells in a patient are provided. Such methods comprise, in some embodiments, administering to the patient an agent selected from a MEK inhibitor, an ERK inhibitor, a Raf inhibitor, a Ras inhibitor, a Syk inhibitor, a p60-c-Src inhibitor, a β-arrestin1/2 inhibitor and a GRK2 inhibitor. In some embodiments, a method comprises administering to the patient or subject, or contacting a sickle red blood cell with, a MEK inhibitor, an ERK inhibitor, a Raf inhibitor, a Ras inhibitor, a Syk inhibitor, a p60-c-Src inhibitor, a β-arrestin1/2 inhibitor and/or a GRK2 inhibitor.

In some embodiments, a method of inhibiting formation of multicellular aggregates in the presence of sickle red blood cells or in a subject with sickle cell disease is provided. The method comprises administering to the patient or subject with sickle cell disease, or contacting, a sickle red blood cell with an agent selected from a MEK inhibitor, an ERK inhibitor, a Raf inhibitor, a Ras inhibitor, a Syk inhibitor, a p60-c-Src inhibitor, β-arrestin1/2 inhibitor and a GRK2 inhibitor.

In some embodiments, a method of inhibiting activation and adhesion of leukocytes to endothelial cells in the presence of sickle red blood cells or in a subject with sickle cell disease is provided. The method comprises administering to the patient or subject with sickle cell disease, or contacting the sickle red blood cells with an agent selected from a MEK inhibitor, an ERK inhibitor, a Raf inhibitor, a Ras inhibitor, a Syk inhibitor, a p60-c-Src inhibitor, a β-arrestin1/2 inhibitor and a GRK2 inhibitor.

In some embodiments, a method of alleviating at least one of acute or chronic pain, chronic hemolysis (aplastic crises), avascular necrosis, organ damage, and erythroid hyperplasia in subjects with sickle cell disease is provided. The method comprises administering to the patient or subject with sickle cell disease, or contacting the sickle red blood cells with an agent selected from a MEK inhibitor, an ERK inhibitor, a Raf inhibitor, a Ras inhibitor, a Syk inhibitor, a p60-c-Src inhibitor, a β-arrestin1/2 inhibitor and a GRK2 inhibitor.

In some embodiments, a method comprises administering to the patient, or contacting a sickle red blood cell with a combination of two or more agents selected from a MEK inhibitor, an ERK inhibitor, a Raf inhibitor, a Ras inhibitor, a Syk inhibitor, a p60-c-Src inhibitor, a β-arrestin1/2 inhibitor and a GRK2 inhibitor. The two or more inhibitors may be co-administered. Co-administration indicates the agents may be administered in any order, at the same time or as part of a unitary composition. The two agents may be administered such that one agent is administered before the other with a difference in administration time of 1 hour, 2 hours, 4 hours, 8 hours, 12 hours, 16 hours, 20 hours, 1 day, 2 days. 4 days, 7 days, 2 weeks, 4 weeks or more.

Administration to a subject may include formulating the therapeutic agents, such as a MEK inhibitor, an ERK inhibitor, a Raf inhibitor, a Ras inhibitor, a Syk inhibitor, a p60-c-Src inhibitor, a β-arrestin1/2 inhibitor and/or a GRK2 inhibitor, with pharmaceutically acceptable carriers and/or excipients, etc., to form pharmaceutical compositions. Suitable formulations for therapeutic compounds are available to those skilled in the art. Administration may be carried out by any suitable method, including intraperitoneal, intravenous, intramuscular, intrathecal, subcutaneous, transcutaneous, oral, nasopharyngeal, or transmucosal absorption among others. The dosage for a particular subject may be determined based on, for example, the subject's weight, height, and/or age; the severity of the subject's disease or symptoms; the length of treatment and/or number of doses anticipated in a particular regiment; the route of administration; etc.

Methods of determining the severity of sickle cell disease or another hemolobinopathy are also provided. The methods include obtaining a blood sample including red blood cells from a subject. The red blood cells may be isolated from the sample for use in the methods or used as whole blood. The red blood cells may be treated with at least one of cholera toxin, pertussis toxin, TNF-α, epinephrine or exposed to hypoxia prior to use in the methods or as a step within the method. The cells are then assessed for expression, activation or membrane translocation of at least one of ERK, Ras, BRAE, Raf1 MEK, β-arrestin 1/2 , Syk, 60-c-Src or GRK2. The cells may be assessed for two, three, four, five, six or more of the markers. Suitably, in this step, the cells are assessed for at least one of ERK phosphorylation and expression, MEK phosphorylation and expression, GRK2 expression and membrane translocation, and phosphorylation, or β-arrestin 1/2 expression and membrane translocation, and phosphorylation. The expression and/or activation levels of these markers, such as the level of ERK phosphorylation and expression, MEK phosphorylation and expression, GRK2 membrane translocation, phosphorylation and expression, or β-arrestin 1/2 membrane translocation, phosphorylation and expression, is related to the severity of sickle cell disease and/or the likelihood of the red blood cells to adhere to other cells and/or to increased endothelial dysfunction and vascular injury or mortality for the subject. Thus red blood cells with increased expression/activation/membrane translocation of any one of or any combination of the listed markers will be more likely to adhere or cause adhesion to the blood vessels or other cells and thus more likely to result in a symptom or morbidity associated with endothelial dysfunction or vascular injury. Increased is a relative term and the comparison may be as compared to normal, non-sickle red blood cells or as a comparison to non-treated red blood cells if the cells were treated with one of the listed agents.

In a still further aspect, methods of treating at least one symptom of a hemoglobinopathy in a subject are provided. The methods include having an expression, activation or membrane translocation level of at least one marker selected from ERK, Ras, BRAF/Raf1, MEK, β-arrestin 1/2, Syk, p60-c-Src, or GRK2 determined in the sample. The level of the marker may be measured after treatment of the cells with cholera toxin, pertussis toxin, TNF-α, epinephrine or exposure of the cells to hypoxia. Based on these levels, a treatment regimen for the subject is selected. The treatment regimen may include a recommendation to administer an agent capable of inhibiting at least one of the markers tested. The agent recommended for administration need not be an agent capable of inhibiting the marker tested or any one of the markers tested and showing increased activation, expression or membrane translocation as compared to a control or reference level, but may be directed at inhibiting any of the indicated markers. The final step includes administering a therapeutically effective amount of an agent capable of inhibiting at least one symptom of the hemoglobinopathy to the subject if the expression and/or activation, and/or membrane-translocation of at least one of ERK, Ras, BRAF/Raf1, MEK, β-arrestin Syk, p60-c-Src, or GRK2 is above that of control cells.

The data provided in the Examples also indicate that the expression and/or activation of ERK, Ras, BRAF/Raf1, MEK, Syk, p60-c-Src, β-arrestin 1/2 or GRK2 in the red blood cells is indicative of the likelihood of adhesion of cells, in particular red blood cells to leukocytes or endothelial cells, and of vascular endothelial injury in the subject and mortality caused by the hemoglobinopathy. The intensity of basal ERK, GRK2, Syk, p60-c-Src and 1t-arrestin1/2 phosphorylation, and the levels of GRK2, Syk, p60-c-Src and β-arrestin1/2 bound to the membrane vary among patients with sickle cell disease and correlate with the severity of disease or appearance of acute crises. Thus, β-arrestin1/2 and GRK2 translocation to the membrane, and phosphorylation and activities as well as the levels of expression of Syk and p60-c-Src and phosphorylation and activities of Syk, p60-c-Src and the other markers can be used as a prognostic tool for sickle cell severity. For this end, screening patients both when they are asymptomatic during steady state and during vaso-occlusive crisis for RBC expression and/or phosphorylation/activation of ERK, Ras, BRA F/Raf1, MEK, Syk, p60-c-Src, -arrestin 1/2 or GRK2 in the red blood cells will provide an opportunity to aggressively treat a patient when the patient is likely to experience an acute crises resulting in symptoms. This will help determine sickle cell severity in subjects and could also help predict precipitation and alleviation of painful vaso-occlusive crisis.

The following examples are illustrative and are not intended to limit the disclosed subject matter. All references cited herein are incorporated herein by reference in their entireties.

### EXAMPLES

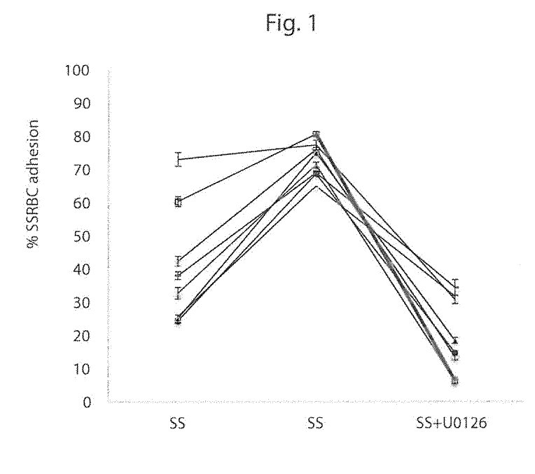

Sickle cell ERK mediates adhesion to endothelium, activation of leukocyte adhesion, and vaso-occlusion. Basal activation. We have previously shown that ERK remains active in sickle red cells but not in normal red cells. To determine if ERK activation mediates sickle red cell adhesion to endothelial cells, we used inhibitors of the ERK kinase, MEK, to repress ERK activity. Adhesion of sickle red cells to endothelial cells was then assessed. Adhesion of sickle red cells to normal endothelial cells was consistent within but not between patients (FIG. 1). Endothelial activation with TFNα resulted in increased sickle cell adhesion, and such increase also varied among patients. Sickle red cells from ˜50% SCD patients exhibited >1.5-fold increase in adhesion. ERK inhibition with U0126 MEK inhibitor reduced sickle cell adhesion (p<0.05) (FIG. 1).

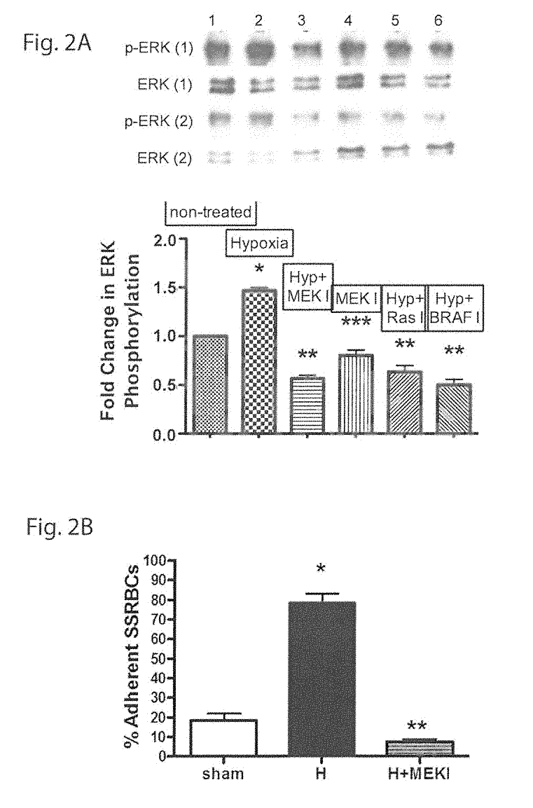

Inducible ERK activation in sickle red cells. One of the major pathophysiologic processes in SCD is vasoocclusion in response to hypoxia. Sickle red cells exposed to hypoxia increased ERK phosphorylation (FIG. 2A). Inhibition of Ras, Raf1 /BRAF and MEK activity by farnesylthiosalicylic acid that disrupts active Ras binding to the membrane, GW5074 and U0126, respectively, decreased the effect of hypoxia on ERK phosphorylation (FIG. 2A), suggesting that Ras, Raf1/BRAF and MEK are involved in increased activation of ERK. Exposure of sickle cells to hypoxia (8% O₂) for 2 hours up-regulated sickle cell adhesion to endothelial cells (FIG. 28). The effect of hypoxia on sickle cell adhesion was almost completely inhibited with the MEK inhibitor RDEA119 (FIG. 2B). These data underscore in pathological conditions the significance of RBC ERK in adhesive contact with the endothelium, and suggest that MEK/ERK signaling in sickle cells can be activated by hypoxic stress to mediate adherence of these cells to the endothelium.

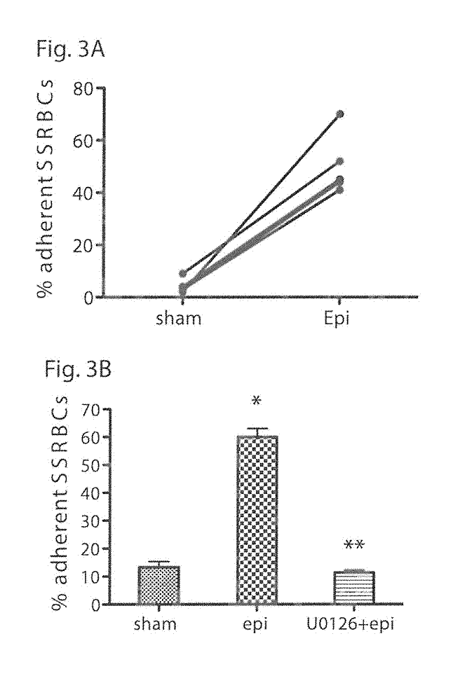

Acute pain crises are unpredictable, but may also be induced by physical, emotional, or psychological stress, supporting a role for adrenergic signaling. Systemic administration of propranolol to SCD patients to block adrenergic signaling, inhibited human sickle red cell adhesion, and the associated vasoocclusion in mice. Epinephrine increased sickle cell adhesion to normal endothelial cells (FIG. 3A and 3B), and the degree of change above basal adhesion varied among sickle cell patients (FIG. 3A). U0126 inhibited activated sickle cell adhesion (FIG. 3B). Thus, ERK signal is required for red cell-endothelial cell binding, and the data suggest that ERK activity level may affect sickle cell adhesion to endothelial cells.

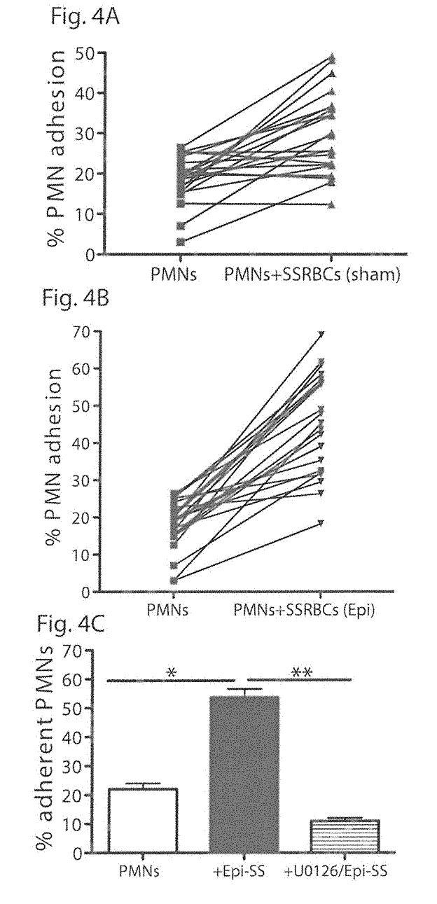

We further show that sickle cells vary between patients in the ability to mediate leukocyte adhesion to endothelial cells (FIG. 4). Sham- or epinephrine-treated sickle red cells co-incubated with naive neutrophils (PMNs) increased PMN adhesion to normal endothelial cells, and the degree of change above basal PMN adhesion varied (FIGS. 4A&B). Sickle cell ERK inhibition with the MEK inhibitor U0126 decreased sickle cell-induced PMN adhesion (FIG. 4C). This suggests that sickle cell ERK activity level may variably affect PMN adhesion.

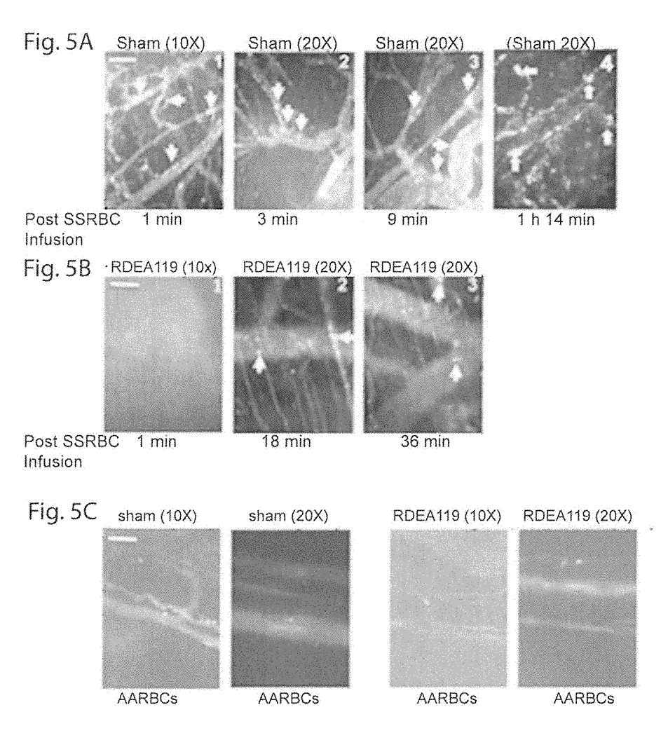

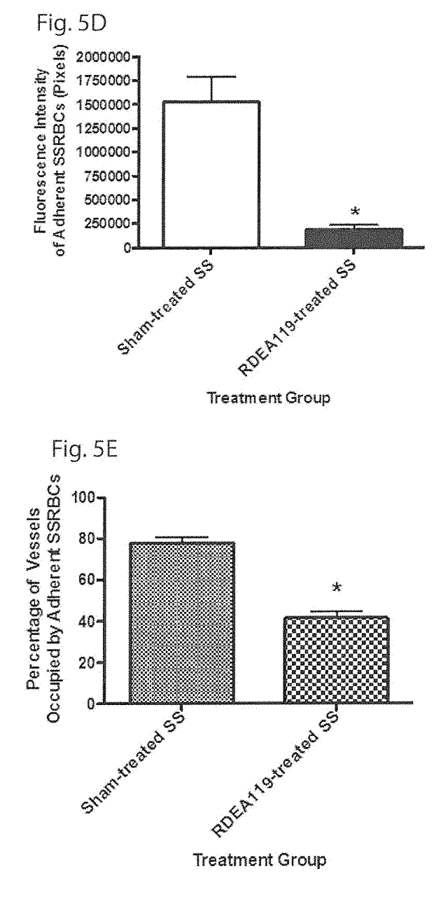

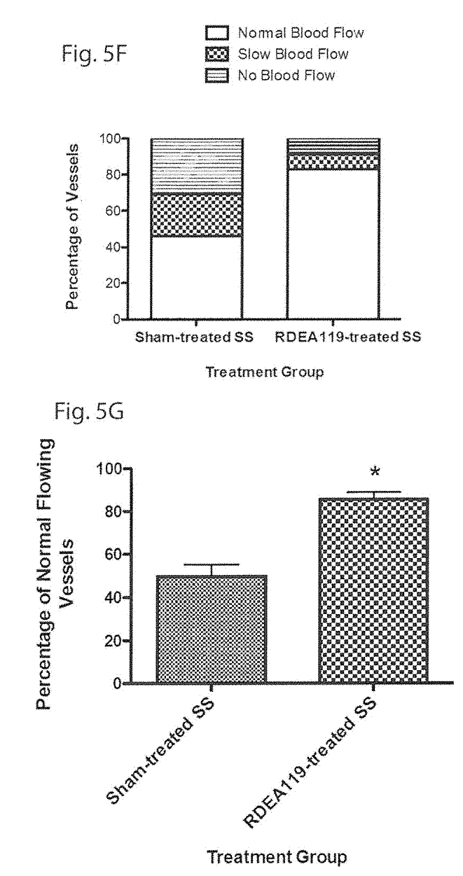

In vivo studies in nude mice. Sickle cell ERK activation is pathophysiologically relevant (FIG. 5). Human sickle red cells were sham-treated or treated with the MEK inhibitor RDEA119 ex vivo, washed then adoptively transferred to nude mice pretreated with TNFα. Intravital microscopy observation of enflamed venules and arterioles visible through the dorsal skin-fold window chamber for at least 1 hour, showed that sham-treated sickle red cells adhered to 78 ±3% of enflamed vessels and arterioles (FIG. 5A and 5D-E). Sickle RBC adhesion occurred progressively occluded micro-vessels with evident blood stasis (FIG. 5A, 5F and 5G). In sharp contrast, RDEA119-treated sickle cells adhered poorly to activated-endothelial cells with no visible vase-occlusion. Adhesion of RDEA 119-treated SSRBCs was reduced by 88% compared to sham-treated SSRBCs (n=5; p <0.0001) (FIG. 5A, 5B and 5D). As a result of RDEA119 treatment, SSRBCs promoted occasional small vessel obstruction, and normal blood flow was restored in 86±3.3% of vessels and arterioles recorded compared to 50±5.5% of vessels with normal blood flow in animals infused with sham-treated cells (p <0.0001; FIG. 5A, 5B, 5F and 5G). The involvement of ERK in normal RBCs adhesion in vivo was also tested. Sham-treated and RDEA119-treated normal human RBCs showed no real adherence in enflamed vessels [FIG. 5C (panels 1, 2, 3 and 4)], further confirming our previous data that ERK is inactive in normal. RBCs. This suggests that targeting ERK can be viable option for reducing vase-occlusion.

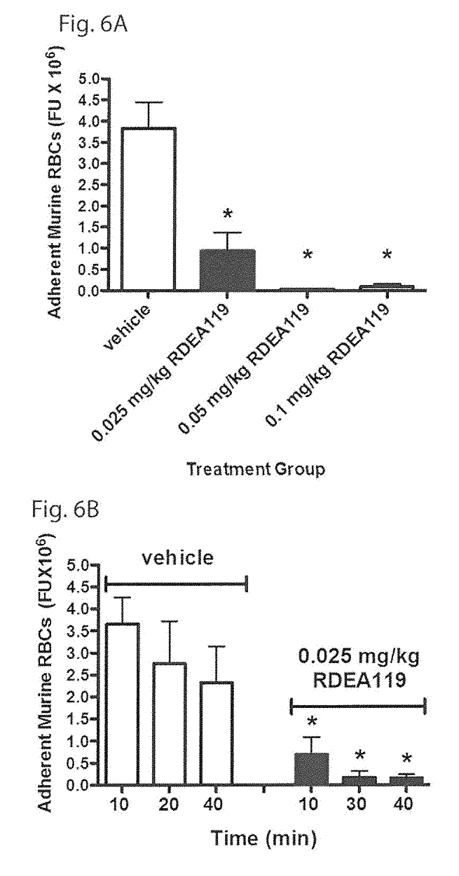

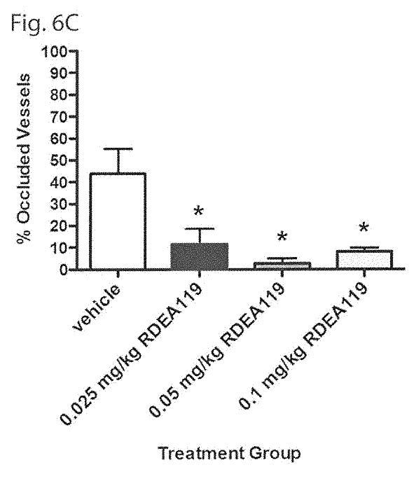

In vivo studies in sickle cell mice. We also evaluated the pathophysiological relevance of ERK in sickle RBCs in transgenic sickle cell mice. We used sickle mice and infused RDEA119 into SCD animals 120 minutes after TNFα injection, a time at which RBCs adhered and a vasoocclusive crisis is established. Murine sickle RBCs adhered markedly in vehicle-treated animals (FIG. 6A). Vasoocclusion occurred in 41±12.4% of vessels, which led thereafter to blood stasis. In contrast, 0.025, 0.05 and 0.1 mg/kg RDEA119 reversed murine sickle RBC adhesion, which was inhibited by 76%, 99% and 98% respectively (p<0.0001 regardless of the dose of RDEA119) (FIG. 6A). RDEA119 even at the lowest dose (0.025 mg/kg) was able to reverse murine sickle RBCs adhesion within the first 10 min of drug administration compared to vehicle (p<0.05) and such effect was sustained over time (FIG. 6B). This led to restored blood flow in 57% of vessels recorded (p<0.05 for each RDEA 119 dose) (FIG. 6C).

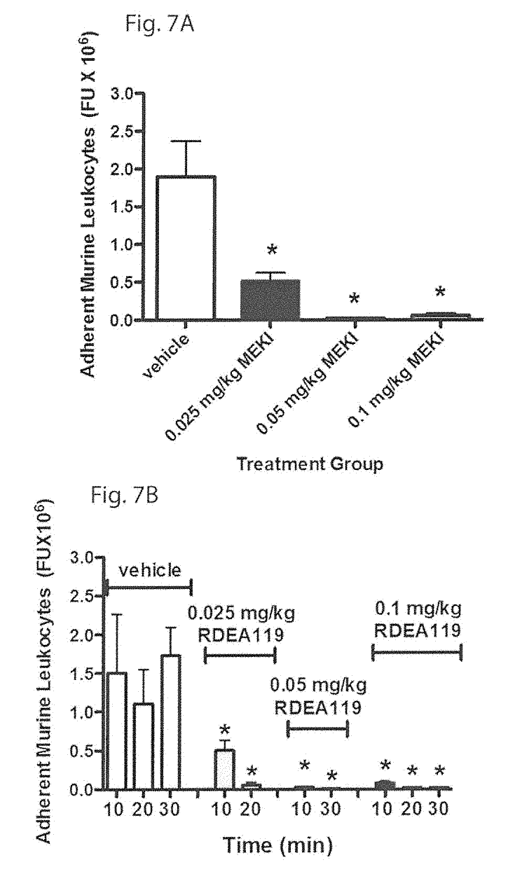

Leukocyte adherence to enflamed venules was also visualized within 10 min in vehicle-treated sickle transgenic mice, and increased slightly over time, promoting vasoocclusion with obvious blood stasis (FIG. 7A). However, 0.025, 0.05 and 0.1 mg/kg MEK inhibitor RDEA 119 reversed leukocyte adhesion, which was reduced by 73%, 99 and 97%, respectively, over a period of 55 min (p<0.001; FIG. 7A). Leukocyte adhesion was abrogated within the first 10 min of 0.025, 0.05 and 0.1 mg/kg RDEA119 administration compared to vehicle treatment (p<0.05), and adhesion further decreased thereafter (p<0.05 regardless of the time following drug administration) (FIG. 78). Together, these data suggest that the anti-adhesive activity of the compound is relatively long, since its action on cell adhesiveness in sickle mice was rapid and persistent, and also provide a proof of principle that ERK is pathophysiologically relevant in SCD, and that inhibition of MEK-dependent ERK activation in sickle RBCs and leukocytes has potential therapeutic benefits in reversing acute vasoocclusive crises.

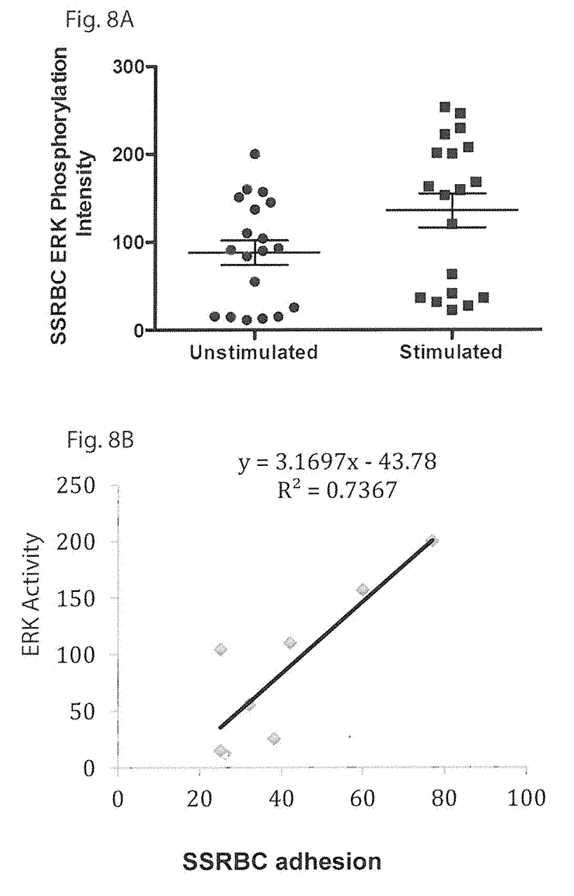

Sickle cell ERK activity level positively correlates with sickle cell adherence to endothelial cells. Membrane protein ghosts were prepared from packed sickle red cells sham-treated or epinephrine-treated. Western blots were then performed using antibodies against phosphorylated and non-phosphorylated ERK. Band densities (Integrated Density) were then measured using ImageJ software and the intensity of ERK phosphorylation was normalized according to the values of non-phosphorylated ERK. As shown in FIG. 8A, sickle cell ERK is phosphorylated at baseline, and ERK phosphorylation levels differ between patients (n=19) (FIG. 8A). Epinephrine increased phosphorylation of sickle cell ERK (n=19; p<0.05), and the level of increase in ERK phosphorylation also varied among patients (FIG. 5A). Similar data were obtained when the activity of ERK was measured.

Sickle RBC adhesion to endothelial cells was determined by calculating % adherent sickle RBCs (SSRBCs) at a shear stress of 2 dynes/cm². ERK kinase activity was determined by immuno-precipitating ERK from sickle RBCs, then co-incubating ERK with its substrate myelin basic protein. Western blots were then performed to detect phosphorylated myelin basic protein, and the intensity of protein phosphorylation was defined by measuring integrated densities of the bands (presented as ERK activity) using the software ImageJ. A correlation was then made to see how ERK activity defined as described above, is related to sickle RBC adhesion. FIG. 8B shows that there is a positive association between the level of ERK activity and % SSRBC adhesion to endothelial cells (r²=0.74,p<0.05, correlation coefficient=0.86), meaning that an increase in ERK activation is related to an up-regulation in sickle RBC adhesion. These data strongly suggest that the elevated basal/inducible ERK activity can cause greater sickle cell adhesive interactions with the endothelium.

Gas protein regulates the protein tyrosine kinases, Src and p72^{Syk}, to mediate sickle cell adhesion to endothelium. Using an Automated Hematology Analyzer, human sickle cell preparations contained 1.02±0.02×10⁶ /μl RBCs, very low levels of contamination with leukocytes (0.4±0.1×10³/μl) in some of the samples tested, and no contamination with platelets (0 cells/μl), making it unlikely that human platelets and the low numbers of leukocytes could affect sickle cell adhesion in our studies.

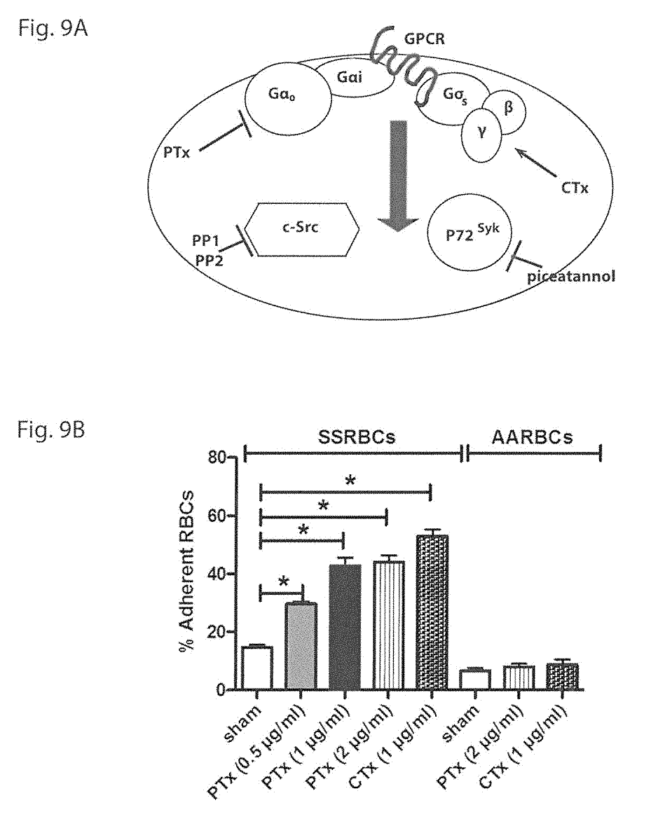

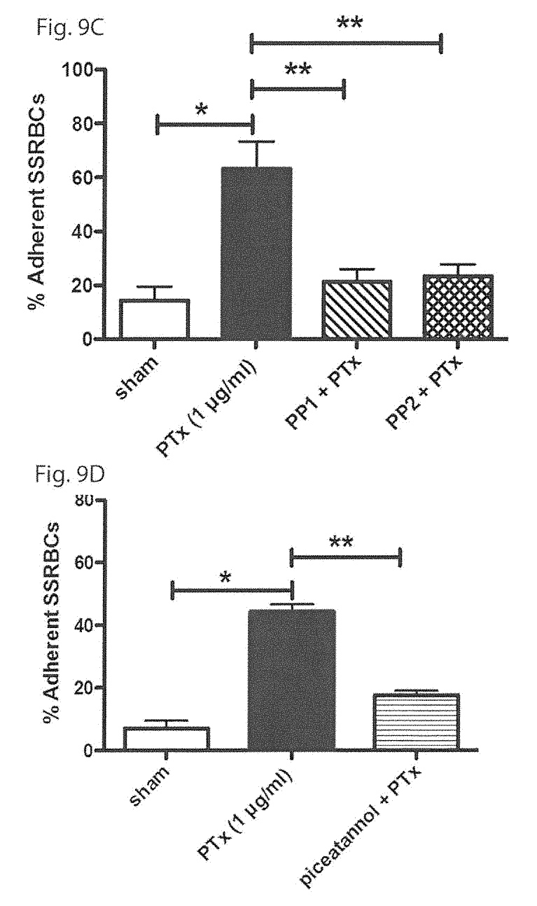

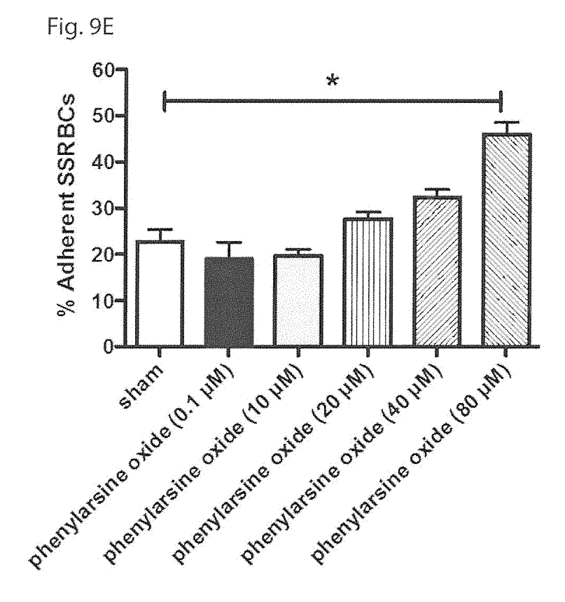

Vaso-occlusion is associated with various types of physiological stress. β2-adrenergic receptors (β2AR) stimulation with catacholamines employs both Gas and &Gaᵢ (or Ga₀) pathways. We first determined the Ga, Gas and/or Gaᵢ, in sickle cell regulating tyrosine kinase activation-mediated sickle cell adhesion to endothelial cells (FIG. 9A). Sickle cells adhered to some degree to endothelial cells under intermittent flow conditions at a shear stress of 2 dynes/cm². (FIGS. 9B-D). However, treatment of sickle cells with 0.5, 1 or 2 μg/ml PTx, an inhibitor of Gai (and Ga₀)-mediated suppression of adenyly! cyclase (FIG. 9A), increased sickle cell adhesion to HUVECs in a dose dependent manner (p <0.001, n=3; FIG. 9B). Similarly, treatment of sickle cells with 1 μg/ml CTx, which directly activates Gas protein (FIG. 9A), also up-regulated their adhesion to endothelial cells (p<0.0001; FIG. 9B). The selective Src-family kinase inhibitors, PP1 and PP2 (FIG. 9A), and the non-receptor tyrosine kinase p72^{Syk} inhibitor, piceatannol (FIG. 9A), inhibited the effect of PTx on sickle cell adhesion by 67±2.7%, 64±1.7% and 60 ±5.5%, respectively (p<0.01) (FIGS. 9C and 9D). In contrast, treatment of normal RBCs (AARBCs) with 2 μg/ml PTx or 1 μg/ml CTx failed to significantly enhance their adhesion to endothelial cells (p>0.05, n=3; FIG. 9B). These data suggest that the protein Gas up-regulates activation of non-receptor Src family and p72^{Syk} tyrosine kinases to mediate SSRBC adhesion to endothelial cells.

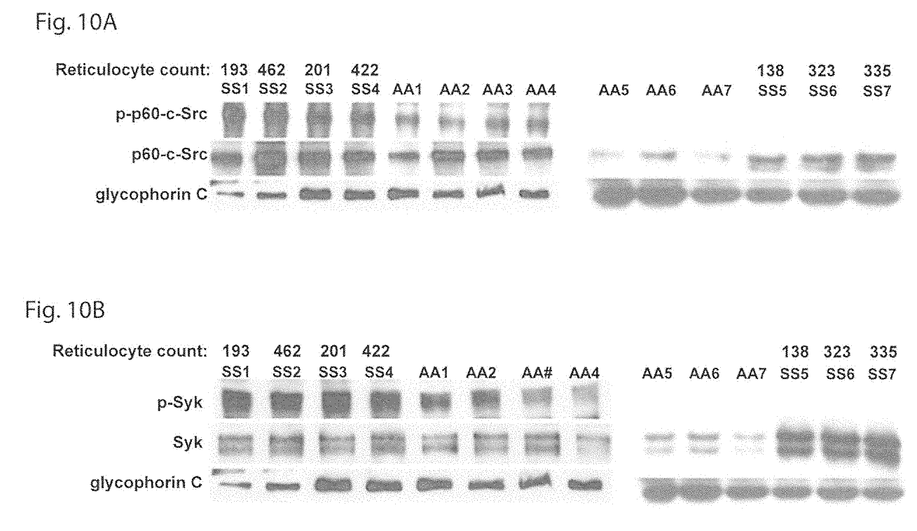

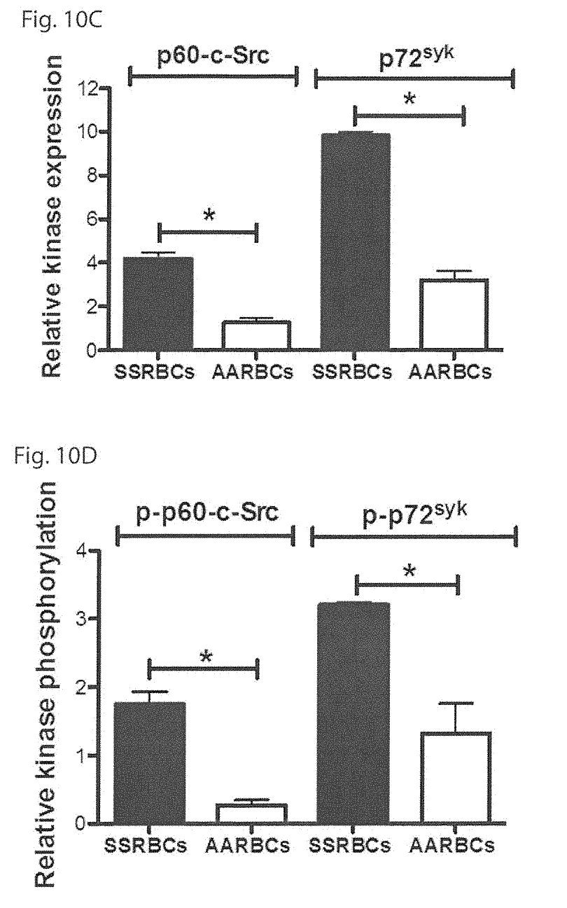

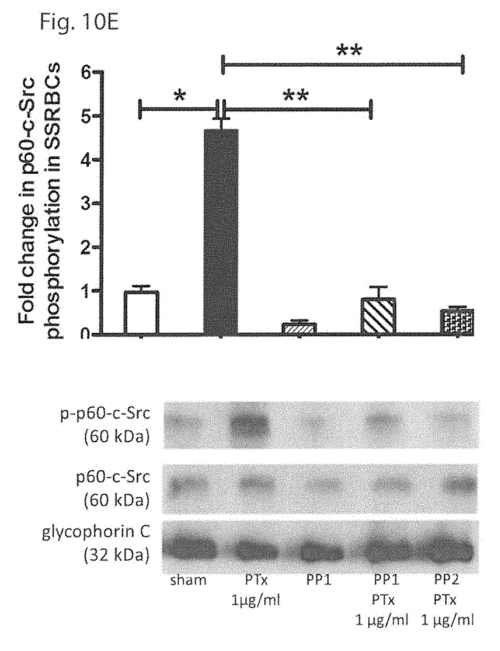

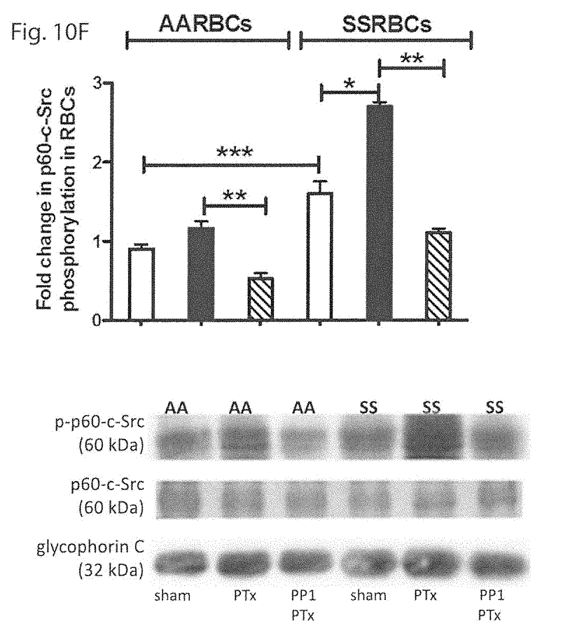

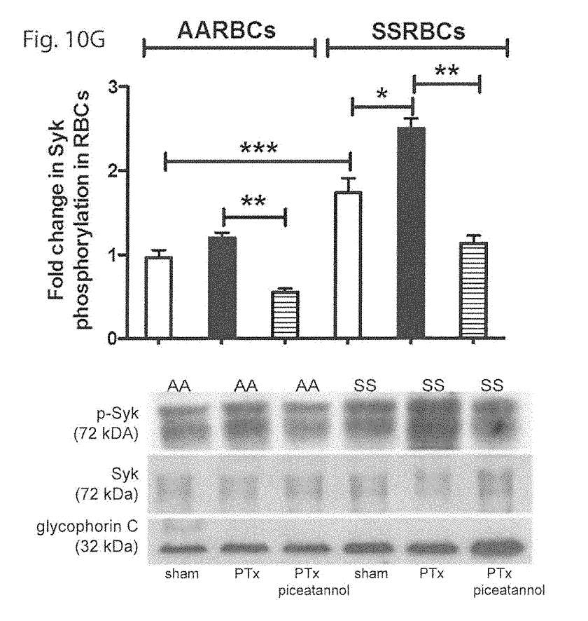

Phosphorylation of the tyrosine kinases p60-c-Src and p72^{Syk} is negatively regulated by Ga₁ (or Ga₀) activation. Given the importance of abnormal sickle cell adherence and its regulation by signaling mechanisms in SCD pathophysiology, we determined whether the Src, p60-c-Src, and p72^{Syk} tyrosine kinases are more active in sickle cell vs. normal cells. The tyrosine kinases p60-c-Src and p72^{Syk} are both expressed and phosphorylated at baseline in normal RBCs and sickle cells (FIGS. 10A and 10B). However, the levels of expression of p60-c-Src and p72^{Syk} are 3.3-fold and 3.1-fold, respectively, higher in sickle cells than in normal RBCs (p<0.001) (FIGS. 10A, 10B and 10C). Similarly, baseline phosphorylation of p60-c-Src (p-p60-c-Src) and p72^{Syk} (p-p72^{Syk}) is 5.7-fold and 2.5-fold, respectively, higher in sickle cells than in normal RBCs (p<0.01) (FIGS. 10A, 10B and 10D). These data suggest that higher baseline phosphorylation levels of p60-c-Src and p72^{Syk} in sickle cells vs normal RBCs is due at least in part to higher kinase expression levels in sickle cells compared to normal RBCs.

Analysis of the data also showed that 1 μg/ml PTx increased significantly p60-c-Src phosphorylation (p<0.0001), which was inhibited by both PP1 and PP2 to levels below baseline phosphorylation (p<0.0001) (FIGS. 10E and 10F). Similarly, PTX treatment of sickle cells also significantly increased phosphorylation of p72^{Syk} (p<0.001) (FIG. 10G). Piceatannol reduced the effect of PTx on p72^{Syk} phosphorylation to levels below baseline phosphorylation (p <0.0001) (FIG. 10G). In contrast, PTx failed to significantly up-regulate phosphorylation of p60-c-Src and p72^{Syk} in normal RBCs (p>0.05). Phosphorylation of p60-c-Src and p72^{Syk} was inhibited with PP1 and piceatannol respectively (FIGS. 10F and 10G). This suggests that in sickle cells, Gai (or Ga₀) activation or PTx itself negatively affects phosphorylation of p60-c-Src and p72^{syk}.

Acute pain crises and organ damage in SCD. While it is clear that survival of SCD patients has improved over the last 40 years, the factors that portend positive and negative prognoses need be readdressed given the development of new treatment modalities. Because SCD patients are now living long enough, in order to give patients and physicians the opportunity to make informed decisions, more detailed data are needed to identify truly favorable and unfavorable phenotypic traits and thus help better identify individualized treatment options for patients with this disease. Clearly, SCD is manifested by diverse, presentations, and its prognosis varies across the patient population. Prior studies have shown renal failure, seizures, acute chest syndrome (ACS), low fetal Hemoglobin level, and baseline white blood cell (WBC) count greater than 15,000 cells per cubic millimeter to be associated with decreased survival.¹

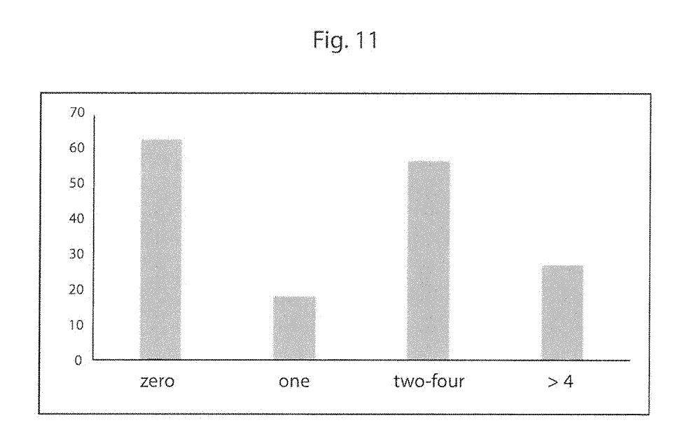

Molecular phenotypes and biomarkers have become a new topic of investigation. To understand the distribution of major complications in our SCD patient population, we collected the following data: I) number of acute pain crises requiring treatment and significant chronic pain requiring daily use of narcotics during the last year; and 2) history of organ injury associated with high morbidity and early death as reflected by organ damage score. Organ damage score was based on the presence or absence of pulmonary dysfunction, kidney dysfunction, central nervous system (CNS) abnormalities, avascular necrosis (AVN) of the hips or shoulders, and leg ulcers. Of 165 patients on narcotics daily (FIG. 11), 38.18% of the patients had no acute crises, 10.91% of the patients had one acute crises, 34.55% of the patients bad 2-4 acute crises and 16.36% of the patients had >4 acute crises. Of 675 patients, 242 patients had an organ damage score of 0, 228 patients had a score of 1, 122. patients had a score of 2, 68 patients had a score of 3, and 15 patients had a score, of 4, and 0 patient has a score of 5, Thus a significant number of patients could benefit from better management of their disease.

### REFERENCES

1. Platt O S, Brambilla D J, Rosse W F, Milner P F, Castro O, Steinberg M H, Klug P P: Mortality in sickle cell disease. Life expectancy and risk factors for early death. N Engl J Med 1994, 330:1639-44.
2. Hebbel R P, Boogaerts M A, Eaton J W, Steinberg M H: Erythrocyte adherence to endothelium in sickle-cell anemia. A possible determinant of disease severity, N Engl J Med 1980, 302:992-995
16. Mohandas N, Evans E: Sickle erythrocyte adherence to vascular endothelium. Morphologic correlates and the requirement for divalent cations and collagen-binding plasma proteins, J Clin Invest 1985, 76:1605-1612
17. Zennadi R, Hines P C, De Castro L M, Cartron J P, Parise L V, Telen M J: Epinephrine acts through erythroid signaling pathways to activate sickle cell adhesion to endothelium via LW-alphavbeta3 interactions, Blood 2004, 104:3774-3781
18. Zennadi R, Chien A, Xu K, Batchvarova M, Telen M J: Sickle red cells induce adhesion of lymphocytes and monocytes to endothelium, Blood 2008, 112:3474-3483
19. Zeimadi R, Moeller B J, Whalen E J, Batchvarova M, Xu K, Shan S. Delahunty M, Dewhirst M W, Telen M J: Epinephrine-induced activation of LW-mediated sickle cell adhesion and vaso-occlusion in vivo, Blood 2007, 110:2708-2717
20. Schmitt J M, Stork P J: beta 2-adrenergic receptor activates extracellular signal-regulated kinases (ERKs) via the small G protein rap 1 and the serine/threonine kinase B-Raf, J Biol Chem 2000, 275:25342-25350
21. Houslay M D, Kolch W: Cell-type specific n of cross-talk between extracellular signal-regulated kinase and cAMP signaling, Mol Pharmacol 2000, 58:659-668
22. Kolch W: Meaningful relationships: the regulation of the Ras/Raf/MEK/ERK pathway by protein interactions, Biochem J 2000, 351 Pt 2:289-305
23. Brzostowski J A, Kimmel A R: Signaling at zero G: G-protein-independent functions for 7-TM receptors, Trends Biochem Sci 2001, 26:291-297
24. Hall R A, Premont R T, Chow C W, Blitzer J T, Pitcher J A, Claing A, Stoffel R H, Barak L S, Shenolikar S, Weinman E J, Grinstein S, Lefkowitz R J: The beta2-adrenergic receptor interacts with the Na+/H+—exchanger regulatory factor to control Na+/H+ exchange, Nature 1998, 392:626-630
25. Belcher J D, Marker P H, Weber J P, Hebbel R P, Vercellotti G M: Activated monocytes sickle cell disease: potential role in the activation of vascular endothelium and vaso-occlusion Blood 2000, 96:2451-2459
26. Fadlon E, Vordermeier S, Pearson T C, Mire-Sluis A R, Dumonde D C, Phillips J, Fishlock K, Brown K A: Blood polymorphonuclear leukocytes from the majority of sickle cell patients in the crisis phase of the disease show enhanced adhesion to vascular endothelium and increased expression of CD64, Blood 1998, 91:266-274
27. Turhan A, Weiss L A, Mohandas N, Coller B S, Frenette P S: Primary role for adherent. leukocytes in sickle cell vascular occlusion: a new paradigm, Proc Natl Acad Sci U S A 2002, 99:3047-3051
28. Jozsef L, Khreiss T, Fournier A, Chan J S, Filep Extracellular signal-regulated kinase plays an essential role in endothelin-1-induced homotypic adhesion of human neutrophil granulocytes, British, journal of pharmacology 2002, 135:1167-1174
37. Zennadi R, Whalen E S, Soderblom E J, Alexander S C, Thompson J W, Dubois L G, Moseley M A, Telen M.1; Erythrocyte plasma membrane-bound ERK 1/2 activation promotes. ICAM-4-mediated sickle red cell adhesion to endothelium, Blood 2012, 119:1217-1227
38. Fukumoto T, Kubota Y, Kitanaka A, Yamaoka G. Ohara-Waki F, Imataki O, Ohnishi H, Ishida T, Tanaka T: Gab 1 transduces PI3K-mediated erythropoietin signals to the Erk pathway and regulates erythropoietin-dependent proliferation and survival of erythroid cells, Cell Signal 2009, 21:1775-1783
39. Jindal H K, Ai Z, Gascard P, Horton C, Cohen C M: Specific loss of protein kinase activities in senescent erythrocytes, Blood 1996, 88:1479-1487
44. Shain W, Forman D S, Madelian V, Turner S N: Morphology of astroglial cells is controlled by beta-adrenergic receptors, J Cell Biol 1987, 105:2307-2314

1. A method of alleviating at least one symptom of a hemoglobinopathy in a subject comprising:
obtaining a sample including red blood cells from a subject;
determining the level of expression and/or activation of at least one marker selected from the group consisting of ERK, Ras, BRAF/Raf1, MEK, β-arrestin1/2, Syk, p60-c-Src, or GRK2 in the sample;
comparing the level of expression, activation, or membrane translocation of the marker to a reference level or control; and
developing a treatment plan for the subject based on the comparison to alleviate at least one symptom of the hemoglobinopathy in the subject.

2. The method of claim 1, further comprising administering an agent capable of inhibiting at least one symptom of the hemoglobinopathy to the subject if the expression and/or activation, and/or membrane-translocation of at least one of ERK, Ras, BRAF/Raf1, MEK, β-arrestin1/2, Syk, p60-c-Src or GRK2 is above that of control cells.

3. The method of claim 1, wherein the hemoglobinopathy is selected from sickle cell disease, β-thalassemia, and hemoglobin H disease.

4. (canceled)

5. The method of claim 1, wherein the at least one symptom is selected from vaso-occlusion, acute or chronic painful episodes, chronic hemolysis (aplastic crises), endothelial dysfunction and injury, avascular necrosis, infection, end-organ damage, erythroid hyperplasia and death.

6. (canceled)

7. (canceled)

8. (canceled)

9. The method of claim 6, wherein administration of the agent inhibits formation of multicellular aggregates in the presence of sickle red blood cells.

10. (canceled)

11. The method of claim 1, further comprising treating the red blood cells with at least one of cholera toxin, pertussis toxin, TNF-α, epinephrine, or exposing the cells to hypoxia (low oxygen tension) prior to the determining step.

12. The method of claim 2, wherein the agent is selected from a MEK inhibitor, an ERK inhibitor, a Raf inhibitor, a GRK2 inhibitor, a β-arrestin 1/2 inhibitor, a Ras inhibitor, a Syk inhibitor, a p60-c-Src inhibitor, propranolol, farnesylthiosalicyclic acid and hydroxyurea.

13. The method of claim 12, wherein the inhibitor is a GRK2 inhibitor optionally selected from a siRNA to GRK2, an antibody to GRK2, and a small molecule inhibitor of GRK2 and PARK1.

14. (canceled)

15. The method of claim 12, wherein the inhibitor is a β-arrestin1/2 inhibitor selected from a siRNA, an antibody and a small molecule inhibitor.

16. The method of claim 12, wherein the inhibitor is a MEK inhibitor selected from U0126, PD98059, PD-334581, GDC-0973, CIP-137401, ARRY-162, ARRY-300, PD318088, PD0325901, CI-1040, BMS 777607, AZD8330, AZD6244, AS703026, RDEA119, and GSK1120212.

17. The method of claim 12, wherein the inhibitor is an ERK inhibitor AEZS-1.

18. The method of claim 12, wherein the inhibitor is a Raf inhibitor selected from sorafenib tosylate, GDC-0879, PLX-4720, regorafenib, PLX-4032, SB-590885-R, RAF265, GW5074, XL281, and GSK2118436.

19. (canceled)

20. The method of claim 1, wherein the subject is human.

21. The method of claim 2, wherein the agent is administered when the level of expression or activation, or membrane-translocation is 1.5 or optionally 2.0 fold or more above that of the control.

22. A method of determining the likelihood of a complication resulting from a hemoglobinopathy comprising obtaining a blood sample comprising red blood cells from a subject; assessing the expression, activation or membrane translocation of a marker selected from ERK, Ras, BRAF/Raf1, MEK, α-arrestin1/2, Syk, p60-c-Src and GRK2 in the cells; and comparing the level of the markers in the sample from the subject to a reference level or control, wherein an increased level of expression, activation or membrane translocation of the marker indicates an increased likelihood of a complication from the hemoglobinopathy.

23.-30. (canceled)

31. A method of treating at least one symptom of a hemoglobinopathy in a subject, comprising having determined an expression, activation or membrane translocation level of at least one marker selected from ERK, Ras, BRAF/Raf1, MEK, β-arrestin1/2, Syk, p60-c-Src, or GRK2 in the sample; selecting a treatment regimen for the subject based on the expression, activation or membrane translocation of at least one of the markers; and administering a therapeutically effective amount of an agent capable of inhibiting at least one symptom of the hemoglobinopathy to the subject if the expression and/or activation, and/or membrane-translocation of at least one of ERK, Ras, BRAF/Raf1, MEK, β-arrestin1/2, Syk, p60-c-Src, or GRK2 is above that of control cells.

32. The method of claim 31, wherein the hemoglobinopathy is selected from sickle cell disease, β-thalassemia, and hemoglobin H disease.

33. The method of claim 31, further comprising treating the red blood cells with at least one of cholera toxin, pertussis toxin, TNF-α, epinephrine or exposing the cells to hypoxia prior to assessing the expression, activation or membrane translocation of the marker.

34. The method of claim 31, wherein the symptom is selected from vaso-occlusion, acute or chronic painful episodes, chronic hemolysis (aplastic crises), endothelial dysfunction and injury, avascular necrosis, infection, end-organ damage, erythroid hyperplasia and death.

35. The method of claim 34, wherein the vaso-occlusion is caused by cellular adhesion in the blood vessels.
